(19) United States
(12) Patent Application Publication
(10) Pub. No.: US 2024/0390521 A1
(43) Pub. Date: Nov. 28, 2024

(54) CHIMERIC MODIFIED ION CHANNELS AND USES THEREOF FOR TREATMENT OF TRIGEMINAL NERVE DISORDERS

(71) Applicant: Kriya Therapeutics, Inc., Morrisville, NC (US)

(72) Inventors: David BLEAKMAN, Morrisville, NC (US); Nino DEVIDZE, Morrisville, NC (US)

(21) Appl. No.: 18/647,670

(22) Filed: Apr. 26, 2024

Related U.S. Application Data
(60) Provisional application No. 63/499,085, filed on Apr. 28, 2023.

(51) Int. Cl. A61K 48/00; A61K 31/4985; A61P 25/04; C07K 14/705; C12N 15/86

(52) U.S. Cl. CPC A61K 48/0058; A61K 31/4985; A61P 25/04; C07K 14/705; C07K 14/70567; C12N 15/86; C12N 2750/14143

(57) ABSTRACT

The present disclosure provides AAV vectors encoding a modified chimeric ligand gated ion channel and methods for the treatment of trigeminal neuralgia in a subject in need thereof.

### CROSS REFERENCE TO RELATED APPLICATIONS

This application claims the benefit of the filing date of U.S. Provisional Patent Application No. 63/499,085, filed Apr. 28, 2023, the contents of which are incorporated herein by reference in their entirety.

### REFERENCE TO SEQUENCE LISTING SUBMITTED ELECTRONICALLY

The content of the electronically submitted sequence listing (Name: 4525_1180001_Seqlisting_ST26; Size: 30,934 bytes; and Date of Creation: Apr. 25, 2024), filed with the application, is incorporated herein by reference in its entirety.

### FIELD OF DISCLOSURE

The present disclosure pertains to nucleic acids and expression vectors to chimeric modified ion channels, and use of the same for treatment of trigeminal nerve disorders including trigeminal neuralgia.

### BACKGROUND

Trigeminal neuralgia (TN), also known as tic douloureux or the suicide disease, is a rare chronic pain condition (Reddy et al. Neurol Clin 32 (2): 539-552, 2014) characterized by recurrent episodes of debilitating sharp, stabbing pain over parts of the face (Zakrzewska et al. BMJ 348: g474, 2014). The intensity of the pain can be physically incapacitating. It is hypothesized that injury or abnormalities in the trigeminal roots or the ganglion result in hyperexcitability of trigeminal axons and cell bodies. Hyperexcitable afferents may give rise to pain paroxysms as a result of synchronized after-discharge neuronal activity.

Ion channels mediate ionic flux in cells, e.g., neurons, where they control electrical signaling within and/or between neurons to influence physiology, sensation, behavior, mood, and cognition.

Ligand gated ion channels (LGICs) have distinct ligand binding properties as well as specific ion conductance properties. For example, nicotinic acetylcholine receptors (nAChRs) bind the endogenous ligand acetylcholine (ACh), which activates conductance for cations and typically depolarizes cells, thereby increasing cellular excitability. In contrast, the glycine receptor (GlyR) binds the endogenous ligand glycine, which activates chloride anion conductance and typically reduces the excitability of cells by hyperpolarization and/or by an electrical shunt of the cellular membrane resistance. Chimeric LGIC can combine a ligand binding domain (LBD) of one LGIC with an ion pore domain (IPD) of another LGIC.

However, in order to use LGICs in the treatment of specific diseases, methods and materials to express LGICs in specific target cells are needed.

### BRIEF SUMMARY

Certain aspects of the disclosure are directed to nucleic acid sequences and vectors comprising LGICs, wherein the LGIC comprises a nucleotide sequence having at least 80% sequence identity to any one of SEQ ID NOs: 1-13, or any sequence listed in Table 1.

In some aspects, a nucleic acid comprises SEQ ID NO: 4, SEQ ID NO: 5, SEQ ID NO: 6, or SEQ ID NO: 7.

In some aspects, a nucleic acid has at least 90% sequence identity to SEQ ID NO: 4, SEQ ID NO: 5, SEQ ID NO: 6, or SEQ ID NO: 7.

In some aspects, a nucleic acid has at least 95% sequence identity to SEQ ID NO: 4, SEQ ID NO: 5, SEQ ID NO: 6, or SEQ ID NO: 7.

In some aspects, a nucleic acid has at least 98% sequence identity to SEQ ID NO: 4, SEQ ID NO: 5, SEQ ID NO: 6, or SEQ ID NO: 7.

In some aspects, a nucleic acid has at least 99% sequence identity to SEQ ID NO: 4, SEQ ID NO: 5, SEQ ID NO: 6, or SEQ ID NO: 7.

In some aspects, a nucleic acid has SEQ ID NO: 4, SEQ ID NO: 5, SEQ ID NO: 6, or SEQ ID NO: 7.

In some aspects, a polynucleotide comprises:
(i) a 5′ inverted terminal repeat (ITR);
(ii) a human synapsin promoter;
(iii) a transgene sequence encoding a Ligand Gated Ion Channel (LGIC) comprising a human α7-nicotinic acetylcholine receptor Ligand Binding Domain (α7-nAChR LBD) and a Glycine Receptor Ion Pore Domain (GlyR IPD);
(iv) a polyA sequence; and
(v) a 3′ ITR.

In some aspects, a polynucleotide further comprises an intron sequence located between the human synapsin promoter and the transgene sequence encoding the LGIC.

In some aspects, provided is a polynucleotide, wherein:
(vi) the 5′ ITR comprises a sequence having at least 90%, 95%, 98%, 99%, or 100% identity to SEQ ID NO: 10;
(vii) the human synapsin promoter comprises a sequence having at least 90%, 95%, 98%, 99% or 100% identity to SEQ ID NO: 11;
(viii) the transgene sequence encodes a polypeptide of SEQ ID NO: 1;
(ix) the poly A sequence comprises a sequence having at least 90%, 95%, 98%, 99%, or 100% identity to SEQ ID NO: 3;
(x) the 3′ ITR comprises a sequence having at least 90%, 95%, 98%, 99%, or 100% identity to SEQ ID NO: 13; and
(xi) optionally, the intron comprises a sequence having at least 90%, 95%, 98%, 99%, or 100% identity to SEQ ID NO: 12.

In some aspects, a polynucleotide comprises a nucleic acid of SEQ ID NO: 4, SEQ ID NO: 6, or SEQ ID NO: 7, which encodes the sequence of SEQ ID NO: 1.

In some aspects, a polynucleotide has at least 90%, 95%, 96%, 97%, 98% or 99% sequence identity to a polynucleotide comprising a nucleic acid of SEQ ID NO: 4, SEQ ID NO: 6, or SEQ ID NO: 7, which encodes the sequence of SEQ ID NO: 1.

Certain aspects of the disclosure are directed to an expression vector comprising a nucleic acid or a polynucleotide described herein.

In some aspects, the expression vector is a viral expression vector.

In some aspects, the vector is an adeno-associated virus (AAV) expression vector.

Certain aspects of the disclosure are directed to a cell comprising an expression vector as described herein.

In some aspects, the cell is an isolated cell in culture.

Certain aspects of the disclosure are directed to an AAV particle comprising an expression vector as described herein and an AAV capsid protein.

In some aspects, the AAV capsid protein is an AAV5 capsid protein.

In some aspects, the AAV capsid protein comprises an amino acid sequence that has at least 90% sequence identity to SEQ ID NO: 9.

In some aspects, the AAV capsid protein comprises an amino acid sequence that has at least 95% sequence identity to SEQ ID NO: 9.

In some aspects, the AAV capsid protein comprises an amino acid sequence that has at least 97% sequence identity to SEQ ID NO: 9.

In some aspects, the AAV capsid protein comprises an amino acid sequence that has at least 98% sequence identity to SEQ ID NO: 9.

In some aspects, the AAV capsid protein comprises an amino acid sequence that has at least 99% sequence identity to SEQ ID NO: 9.

In some aspects, the AAV capsid protein has an amino acid sequence of SEQ ID NO: 9.

Certain aspects of the disclosure are directed to a composition comprising a nucleic acid as described herein, an expression vector as described herein, or an AAV particle as described herein.

In some aspects, the composition further comprises a non-ionic co-polymer.

In some aspects, the non-ionic co-polymer is a poloxamer.

In some aspects, the non-ionic co-polymer is poloxamer 188.

In some aspects, the composition comprises about 0.0005% to about 0.005% poloxamer 188.

In some aspects, the composition comprises about 0.001% poloxamer 188.

In some aspects, the composition further comprises sodium phosphate buffer.

In some aspects, the composition comprises about 1 mM to about 10 mM sodium phosphate buffer.

In some aspects, the composition comprises about 10 mM sodium phosphate buffer.

In some aspects, the composition further comprising sodium chloride.

In some aspects, the composition comprises about 120 mM to about 240 mM sodium chloride.

In some aspects, the composition comprises about 180 mM sodium chloride.

In some aspects, the composition has a pH of about 6.8 to about 7.8.

In some aspects, the composition has a pH of 7.3±0.2.

In some aspects, the composition comprises the AAV particle at about 0.8×10¹³ vg/ml to about 5.0×10¹³ vg/ml.

In some aspects, the composition comprises the AAV particle at about 2×10⁹ vg/ml to about 2×10¹⁰ vg/ml.

Certain aspects of the disclosure are directed to a method of treating a disease or condition caused by hyperexcitability of the trigeminal nerve, the method comprising administering the composition as described herein to a subject in need thereof.

In some aspects, the disease or condition caused by hyperexcitability of the trigeminal nerve is trigeminal neuralgia, a trigeminal autonomic cephalgia, an episodic cluster headache, a chronic cluster headache; trigeminal deafferation pain; burning mouth syndrome; or post-traumatic trigeminal neuropathic pain.

In some aspects, the disease or condition is SUNCT or SUNA.

In some aspects, the disease or condition is trigeminal neuralgia.

In some aspects, the method further comprises administering a small molecule agonist to the subject.

In some aspects, the small molecule agonist is varenicline.

In some aspects, the composition is parenterally administered to the subject.

In some aspects, the small molecule agonist is orally administered to the subject.

In some aspects, the composition is administered by percutaneous injection.

In some aspects, the composition is administered by percutaneous injection into a trigeminal ganglion.

In some aspects, the method comprises administering about 6×10⁷, 2×10⁸, 6×10⁸, 2×10⁹, 6×10⁹, or 2×10¹⁰ AAV particles by percutaneous injection into a trigeminal ganglion.

In some aspects, the method comprises administering about 2×10⁹ to about 2×10¹⁰ AAV particles by percutaneous injection into a trigeminal ganglion.

In some aspects, the method further comprises orally administering 0.5 mg or 1 mg varenicline to the subject.

In some aspects, the composition is administered prior to varenicline.

In some aspects, the composition is administered together with varenicline.

In some aspects, the composition is administered after varenicline.

In some aspects, the subject has acute trigeminal neuralgia.

In some aspects, the subject has chronic trigeminal neuralgia.

In some aspects, varenicline is administered between about 7 days and about 31 days after the administration of the composition.

In some aspects, varenicline is administered about 2 weeks after the administration of the composition.

In some aspects, the composition is administered once and varenicline is administered more than once.

In some aspects, the composition is administered once and varenicline is administered once a day for between about 2 days to about 120 days.

In some aspects, the composition is administered once and varenicline is administered once a day until the subject does not experience trigeminal neuralgia.

In some aspects, the method comprises administering from about 1.85×10⁹ AAV vector genomes to about 1.85×10¹¹ AAV vector genomes to the subject.

### BRIEF DESCRIPTION OF THE DRAWINGS/FIGURES

FIG. 1A shows a schematic of an AAV expression vector containing AAV2 inverted terminal repeats (ITRs) and an expression cassette encoding a human synapsin promoter for driving neuronal expression of the gene for a human chimeric ion channel comprised of a modified (m) nicotinic α7 subtype receptor binding domain and glycine channel ion pore domain (m-α7-GlyR, also called PSAM4-GLyR). FIG. 1B shows a map of a plasmid containing the AAV expression vector of FIG. 1A, origins of replication, and a kanamycin resistance gene.

FIG. 2A shows the escape threshold (ET) in male rats treated with IoN-CCI surgery, AAV5-m-α7-GlyR LGIC, and/or varenicline. FIG. 2B shows the escape threshold (ET) in female rats treated with IoN-CCI surgery, AAV5-m-α7-GlyR LGIC, and/or varenicline.

FIG. 3A shows the escape threshold in male versus female rats treated with ION-CCI

surgery, AAV5-m-α7-GlyR LGIC, and/or varenicline. FIG. 3B shows the escape threshold data averaged between male and female rats.

FIG. 4A shows the male/female average escape threshold data at three dose levels when IoN-CCI was performed prior to AAV5-m-α7-GlyR LGIC treatment. FIG. 4B shows the male/female average escape threshold data when IoN-CCI was performed after AAV5-m-α7-GlyR LGIC treatment.

FIG. 5 shows the average male/female escape threshold data with intermittent varenicline in rats pretreated with AAV5-m-α7-GlyR LGIC.

FIG. 6A shows the percentage of trigeminal ganglia with gene of interest (GOI) mRNA ipsilateral to AAV5-m-α7-GlyR LGIC administration. FIG. 6B shows the percentage of trigeminal ganglia with viral DNA ipsilateral to AAV5-m-α7-GlyR LGIC. FIG. 6C shows the percentage of trigeminal ganglia with gene of interest (GOI) mRNA contralateral to AAV5-m-α7-GlyR LGIC administration. FIG. 6D shows the percentage of trigeminal ganglia with viral DNA contralateral to AAV5-m-α7-GlyR LGIC administration. FIG. 6E shows relative viral DNA (bars) and escape threshold (dots) for IoN-CCI surgery followed by AAV5-m-α7-GlyR LGIC administration and AAV5-m-α7-GlyR LGIC administration prior to IoN-CCI surgery at different dose levels of AAV5-m-α7-GlyR LGIC. FIG. 6F shows gene expression (bars) and escape threshold (dots) for ION-CCI surgery followed by AAV5-m-α7-GlyR LGIC administration and AAV5-m-α7-GlyR LGIC administration prior to IoN-CCI surgery at different dose levels of AAV5-m-α7-GlyR LGIC.

FIG. 7A shows the concentration of varenicline in jugular vein blood (JV) following oral administration of three different doses of varenicline. FIG. 7B shows the concentration of varenicline in prefrontal cortex (PFC) following oral administration of three different doses of varenicline. FIG. 7C shows the concentration of varenicline in cerebrospinal fluid (CSF) following oral administration of three different doses of varenicline in serial samples. FIG. 7D shows the concentration of varenicline in cerebrospinal fluid (CSF) following oral administration of three different doses of varenicline in terminal samples (after 720 min).

FIG. 8 shows the biodistribution of AAV5-m-α7-GlyR LGIC at the highest assessed dose with and without varenicline.

### DETAILED DESCRIPTION

Certain aspects of the disclosure are directed to materials and methods for modulating neuron activity in trigeminal neurons by introducing an AAV vector encoding a modified chimeric ligand gated ion channel (LGIC) with increased sensitivity to an exogenous ligand and/or reduced sensitivity to an endogenous ligand into a subject in need thereof.

Certain aspects of the disclosure are directed to a modified LGIC comprises at least one modified LGIC subunit having a ligand binding domain (LBD) and an ion pore domain (IPD), and having at least one modified amino acid (e.g., an amino acid substitution) that confers pharmacological selectivity for binding an exogenous ligand.

Certain aspects of the disclosure are directed to methods of treating neuralgias associated with the trigeminal nerve, more specifically neuralgias caused by hyperexcitability of the trigeminal nerve, including trigeminal neuralgia, and trigeminal autonomic cephalgias (“TACs”) including short-lasting unilateral neuralgiform headache attacks with conjunctival injection and tearing (“SUNCT”) and short-lasting unilateral neuralgiform headache attacks with cranial autonomic symptoms (“SUNA”), episodic cluster headache, and chronic cluster headache; trigeminal deafferation pain; burning mouth syndrome; or post-traumatic trigeminal neuropathic pain by administering an AAV5 vector encoding a modified LGIC that selectively binds varenicline to a subject having trigeminal neuralgia and further administering varenicline to the subject.

Certain aspects of the disclosure are directed to methods of modulating the excitability of a neuronal cell in a subject by administering an AAV5 vector encoding a modified LGIC that selectively binds varenicline to a subject having a condition or disorder caused by neuronal cell excitability.

Certain aspects of the disclosure are directed to methods of modulating the activity of a neuronal cell in a subject, wherein activity of a neuronal cell includes, but is not limited to, active transport (e.g., ion transport), passive transport, excitation, ion flux (e.g., calcium ion flux), and exocytosis by administering an AAV5 vector encoding a modified LGIC that selectively binds varenicline to a subject having a condition or disorder caused by any of the described neuronal cell activities.

Certain aspects of the disclosure are directed to methods of modulating ion transport across a neural cell membrane of a subject by administering an AAV5 vector encoding a modified LGIC that selectively binds varenicline to a subject having ion transport across a neural cell membrane.

Certain aspects of the disclosure are directed to nucleic acids and polynucleotides comprising a modified chimeric LGIC disclosed herein or a subunit thereof.

Certain aspects of the disclosure are directed to expression constructs comprising any of the modified chimeric LGIC disclosed herein of a subunit thereof.

Certain aspects of the disclosure are directed to a viral particle comprising any of the modified chimeric LGIC disclosed herein or a subunit thereof.

Certain aspects of the disclosure are directed to a pharmaceutical composition comprising any of the modified chimeric LGIC disclosed herein or a subunit thereof.

Certain aspects of the disclosure are directed to a cell comprising any of the modified chimeric LGIC disclosed herein or a subunit thereof.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order that the present disclosure can be more readily understood, certain terms are first defined. Additional definitions are set forth throughout the detailed disclosure.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

“Nucleic acid,” “polynucleotide,” and “oligonucleotide,” are used interchangeably in the present application. These terms refer only to the primary structure of the molecule. Thus, these terms include double- and single-stranded DNA, as well as double-and single-stranded RNA. The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

The term “mRNA,” as used herein, refers to a single stranded RNA that encodes the amino acid sequence of one or more polypeptide chains.

As used herein, the term “promoter” refers to a DNA sequence recognized by the machinery of the cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible by external signals or agents; such elements can be located in the 5′ or 3′ regions of the native gene. In some aspects, the promoter is a synapsin promoter that promotes expression of an operably linked gene in a cell of the nervous system.

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

The terms “transcriptional regulatory protein,” “transcriptional regulatory factor,” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA can be indirect by way of binding to another protein that in turn binds to, or is bound to a DNA response element.

As used herein, the term “termination signal sequence” can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “poly A site,” i.e., a site on a RNA transcript to which adenine residues will be added by post-transcriptional polyadenylation.

As used herein, the term “internal ribosome entry site” or “IRES” refers to an element that promotes direct internal ribosome entry to the initiation codon of a cistron (a protein encoding region), thereby leading to the cap-independent translation of the gene. See, e.g., Jackson R J et al., Trends Biochem Sci 15 (12): 477-83 (199); Jackson R J and Kaminski, A. RNA 1(10): 985-1000 (1995). “Under translational control of an IRES” means that translation is associated with the IRES and proceeds in a cap-independent manner.

The term “multicistronic” or “multicistronic vector” refers to a nucleic acid sequence having two or more open reading frames (e.g., genes). An open reading frame in this context is a sequence of codons that is translatable into a polypeptide or protein (e.g. a heavy chain or a light chain). “Bicistronic” or “bicistronic vector” refers to a nucleic acid sequence having two open reading frames (e.g., genes). An open reading frame in this context is a sequence of codons that is translatable into a polypeptide or protein (e.g. a heavy chain or a light chain). In some aspects, the construct of the disclosure is a multicistronic (e.g., bicistronic) construct (e.g., comprising a LBD and a IPD).

The term “self-processing cleavage site” or “self-processing cleavage sequence,” as used herein refers to a post-translational or co-translational processing cleavage site or sequence. Such a “self-processing cleavage” site or sequence refers to a DNA or amino acid sequence, e.g., a 2A site, sequence or domain or a 2A-like site, sequence or domain. The term “self-processing peptide” is defined as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

The term “additional proteolytic cleavage site,” refers to a sequence that is incorporated into an expression construct adjacent a self-processing cleavage site, such as a 2A or 2A like sequence, and provides a means to remove additional amino acids that remain following cleavage by the self-processing cleavage sequence. Exemplary “additional proteolytic cleavage sites” include, but are not limited to, furin cleavage sites with the consensus sequence RXK(R)R. Such furin cleavage sites can be cleaved by endogenous subtilisin-like proteases, such as furin and other serine proteases within the protein secretion pathway. In some aspects, other exemplary “additional proteolytic cleavage sites” can be used, as described in e.g., Lie et al., Sci Rep 7, 2193 (2017).

The terms “operatively linked,” “operatively inserted,” “operatively positioned,” “under control” or “under transcriptional control” means that a promoter is in the correct location and orientation in relation to a nucleic acid to control RNA polymerase initiation and expression of a gene and/or the molecule encoded by a nucleic acid of interest. The term “operably linked” means that a DNA sequence and a regulatory sequence(s) are connected in such a way as to permit gene expression and/or expression of the molecule encoded by a nucleic acid of interest when the appropriate molecules (e.g., transcriptional activator proteins) are bound to the regulatory sequence(s).

As used herein, the terms “nucleic acid of interest”, “polynucleotide of interest”, and the like, generally refer any one or more nucleic acid sequences that encode one or more corresponding molecules, e.g., proteins, whose expression is desired, e.g., modified LGIC proteins. In some aspects, the nucleic acid of interest are selected for placement into a construct and/or delivery vector, e.g., a vector construct, e.g., a viral vector construct as described herein. In some aspects, the nucleic acid of interest can be any gene sequence or functional portion thereof from any organism.

A “coding sequence” or a sequence “encoding” a particular molecule (e.g., a protein) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

As used herein, the terms “backbone” and “backbone polynucleotide” refers to a polynucleotide sequence of a polynucleotide-based vector or plasmid, which does not include the transgene, regulatory elements for the transgene, or terminal repeat sequences. As used herein, a backbone does not include a promoter, an open reading frame comprising a polynucleotide of interest, a polyA tail, or terminal repeat sequences. Backbone polynucleotides can be engineered to encode selectable markers or reporters that provide for the selection or identification of cells that have incorporated the polynucleotide-based vector. Expression of selectable markers or reporters allows identification and/or selection of host cells that incorporate and express other coding regions contained on the polynucleotide-based vector.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., an AAV vector) that is derived from a second nucleic acid sequence (e.g., another AAV vector) can include a nucleotide sequence that is identical or substantially similar to the nucleotide sequence of the second nucleic acid sequence.

In some aspects of the polynucleotides described herein, the derived species can be obtained by, for example, naturally occurring mutagenesis, artificial directed mutagenesis or artificial random mutagenesis. The mutagenesis used to derive polynucleotides can be intentionally directed or intentionally random, or a mixture of each. The mutagenesis of a polynucleotide to create a different polynucleotide derived from the first can be a random event (e.g., caused by polymerase infidelity) and the identification of the derived polynucleotide can be made by appropriate screening methods.

As used herein, the term “mutation” refers to any changing of the structure of a gene, resulting in a variant (also called “mutant”) form that can be transmitted to subsequent generations. Mutations in a gene can be caused by the alternation of single base in DNA, or the deletion, insertion, or rearrangement of larger sections of genes or chromosomes.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values can be generated using the sequence comparison computer program BLAST.

The term “conservative amino acid substitution,” as used herein, refers to a substitution of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Further, a predicted nonessential amino acid residue in an antigen binding arm can be replaced with another amino acid residue from the same side chain family.

An amino acid substitution can include but is not limited to the replacement of one amino acid in a polypeptide with another amino acid. Conservative amino acid substitutions can encompass non-naturally occurring amino acid residues, which are typically incorporated by chemical peptide synthesis rather than by synthesis in biological systems. Naturally occurring residues can be divided into classes based on common side chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

Non-conservative substitutions involve exchanging a member of one of these classes for another class. For example, glycerin can be mutated to alanine, aspartic acid to asparagine or alanine, or tyrosine to alanine.

As used herein, the term “modified” refers to a changed state or structure of a molecule of the disclosure. Molecules can be modified in many ways including chemically, structurally, and functionally. In some aspects, the modification is relative to a reference wild-type molecule.

The term “modified ligand gated ion channel,” as used herein refers to a ligand gated ion channel that includes at least one mutation in either a ligand binding domain or a ion pore domain or both.

The term “chimeric ligand gated ion channel,” as used herein, refers to a ligand gated ion channel that combines portions of different ligand gated ion channels. For example, a chimeric ligand gated ion channel can be a non-naturally occurring combination of a ligand binding domain from a first ligand gated ion channel and an ion pore domain from a second ligand gated ion channel.

As used herein, the term “synthetic” means produced, prepared, and/or manufactured by the hand of man. Synthesis of polynucleotides or polypeptides or other molecules of the present disclosure can be chemical or enzymatic.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components, e.g., an albumin domain, to increase half-life.

As used herein, the term “delivery vector” or “vector” refers to any vehicle for the cloning of and/or transfer of a nucleic acid into a host cell, such as a plasmid, phage, transposon, cosmid, chromosome, artificial chromosome, virus, virion, etc. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “delivery vector” or “vector” includes both viral and nonviral vehicles for introducing the nucleic acid into a cell in vitro, ex vivo or in vivo.

The term “expression vector or construct” means any type of genetic construct containing a nucleic acid in which part or all of the nucleic acid encoding sequence is capable of being transcribed.

A “viral vector” refers to a vector created from at least part of a viral genome which can be used to carry or deliver one or more polynucleotide regions encoding or comprising a molecule of interest, e.g., a protein or a plurality of proteins. Viral vectors can be used to deliver genetic materials into cells. Viral vectors can be modified for specific applications. In some aspects, the delivery vector of the disclosure is a viral vector selected from the group consisting of an adeno-associated viral (AAV) vector, an adenoviral vector, a lentiviral vector, or a retroviral vector. In some aspects, the viral vector is an AAV vector.

The term “adeno-associated virus vector” or “AAV vector” as used herein refers to any vector that comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, bovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2 chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome” and “AAV vector” can be used interchangeably. In some aspects, the AAV vector is modified or mutated relative to the wild-type AAV serotype sequence. In some aspects, the AAV vector is an AAV5 vector comprising an AAV5 capsid encapsulating a vector genome comprising AAV2 inverted terminal repeats (ITRs).

As used herein, the term “AAV particle” or “AAV virion” are used interchangeably and generally refer to an AAV virus that comprises an AAV capsid encapsulating an AAV vector having at least one payload region (e.g., a polynucleotide of interest) and at least one ITR region.

The term “varenicline,” as used herein, refers to a 6,10-Methano-6H-pyrazino[2,3-h][3]benzazepine, 7,8,9,10-tetrahydro-5,8,14-triazatetracyclo[10.3.1.02,11.04,9]hexadeca-2,4,6,8,10-pentaene tartrate salt.

The term “non-ionic co-polymer,” as used herein, refers to a molecule comprising at least two polymer components that are arranged so as to form a co-polymer that does not contain a charge.

The term “poloxamer,” as used herein, refers to a block co-polymer consisting of a hydrophobic chain of poly(propylene oxide) (PPO) flanked by two blocks of hydrophilic poly(ethylene oxide) (PEO). For poloxamer 188, e.g., the PPO chain contains a unit number ranging from 25 to 30, and each PEO block is composed of 75 to 85 EO units in average.

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large and includes, e.g., siRNA, shRNA, sense and/or anti-sense sequences, DNA encoding one or more genes and organized into an expression plasmid, e.g., a vector.

As used herein, the term “administration” refers to the administration of a composition of the present disclosure (e.g., a viral vector (e.g., AAV vector), an AAV particle, or a gene therapy composition) to a subject or system. Administration to an animal subject (e.g., to a human) can be by any appropriate route, such as intramuscular, intravenous, or to a trigeminal ganglion.

As used herein, the term “subject” refers to any organism to which a composition disclosed herein, e.g., an AAV vector of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

The terms “treat,” “treated,” and “treating,” as used herein, mean therapeutic treatment measures wherein the object is to slow down or lessen an undesired physiological condition, disorder, or disease, or obtain beneficial or desired clinical results. In some aspects, treating reduces or lessens the symptoms associated with a disease or disorder. In some aspects, the treating results in a beneficial or desired clinical result, e.g., reduction or abrogation of trigeminal neuralgia.

Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. In some aspects, treatment includes eliciting a clinically significant response without excessive levels of side effects.

As used herein, the term “amelioration” or “ameliorating” refers to a lessening of severity of at least one indicator of a condition or disease, e.g., a trigeminal pain.

As used herein, the terms “effective amount,” “therapeutically effective amount,” and a “sufficient amount” of, e.g., a polynucleotide, expression cassette, vector, AAV particle, or composition of the disclosure refer to a quantity sufficient, when administered to a subject, including a human, to effect beneficial or desired results, including clinical results, and, as such, an “effective amount” or synonym thereto depends on the context in which it is being applied. In some aspects, a therapeutically effective amount of an agent (e.g., an AAV vector, an AAV capsid, a gene therapy composition) is an amount that results in a beneficial or desired result in a subject, e.g., a reduction or abrogation of trigeminal neuralgia as compared to a control.

The amount of a given agent (e.g., a polynucleotide, expression cassette, vector, AAV particle, or composition) will vary depending upon various factors, such as the given agent, the pharmaceutical formulation, the route of administration, the type of disease or disorder, the identity of the subject (e.g., age, sex, and/or weight) or host being treated, and the like.

As used herein, the term “preventing” or “prevention” refers to prophylactic or preventative measures wherein the object is to prevent an undesired physiological condition, disorder, or disease from occurring, delaying or forestalling the onset, development or progression of a condition or disease for a period of time, including weeks, months, or years.

The term “prophylactically effective amount,” as used herein, includes the amount of an agent, (e.g., an AAV vector, an AAV capsid, or a gene therapy composition) that, when administered to a subject having or predisposed to have a disease or disorder (e.g., trigeminal neuralgia) effects beneficial or desired results. Ameliorating a disease or disorder includes slowing the course of the disease or disorder or reducing the severity of later-developing disease or disorder. The “prophylactically effective amount” can vary depending on the characteristics of the agent, e.g., a polynucleotide, expression cassette, vector, AAV particle, or composition, how the agent is administered, the degree of risk of disease, and the history, age, weight, family history, genetic makeup, the types of preceding or concomitant treatments, if any, and other individual characteristics of the subject to be treated.

As used herein, the term “gene therapy” is the insertion of nucleic acid sequences (e.g., a polynucleotide comprising a promoter operably linked to a nucleic acid encoding a therapeutic molecule as disclosed herein) into an individual's cells and/or tissues to treat, reduce the symptoms of, or reduce the likelihood of a disease.

As used herein, “off target” refers to any unintended effect on any one or more target, gene, or cellular transcript.

As used herein, the term “in vitro” refers to events that occur in an artificial environment, e.g., in a test tube or reaction vessel, in cell culture, in a Petri dish, etc., rather than within an organism.

As used herein, the term “in vivo” refers to events that occur within an organism (e.g., animal, human, tissue or cell thereof).

As used herein, the term “ION CCI” or infraorbital nerve (IoN) chronic constriction injury (CCI) refers to an injury induced by surgical constriction of the infraorbital nerve to induce mechanical allodynia. The ION-CCI model is an art-recognized model of trigeminal neuralgia.

As used herein, the term “escape threshold” or “ET” refers to the level of mechanical pressure an animal withstands before withdrawing or escaping from the pain or discomfort of the mechanical stimulus. The stimulation can be achieved, e.g., using von Frey filaments, which are small pieces of nylon rods of varying diameters with which an animal is contacted. For example, the escape threshold is the threshold at which a rat having undergone surgery for chronic infraorbital nerve constriction withdraws from the pain or discomfort of a mechanical stimulus exerted at, or close to, the injured area by von Frey filaments.

By “level” is meant a level or activity of a protein, or mRNA encoding the protein, optionally as compared to a reference. The reference can be any useful reference, as defined herein. By a “decreased level” or an “increased level” of a protein is meant a decrease or increase in protein level, as compared to a reference. A level of a protein can be expressed in mass/vol (e.g., g/dL, mg/mL, μg/mL, ng/mL) or percentage relative to total protein or mRNA in a sample.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a composition described herein; a sample from a subject that has been treated by a composition described herein; or a sample of a purified protein (e.g., any described herein) at a known normal concentration.

The term “pharmaceutical composition,” as used herein, represents a composition comprising a compound or molecule described herein, e.g., an AAV vector disclosed herein, formulated with a pharmaceutically acceptable excipient, and can be manufactured or sold with the approval of a governmental regulatory agency as part of a therapeutic regimen for the treatment of disease in a mammal.

A “pharmaceutically acceptable excipient,” as used herein, refers to any ingredient other than the compound or vector described herein (for example, a vehicle capable of suspending or dissolving the active compound or vector) and having the properties of being substantially nontoxic and non-inflammatory in a subject.

### II. Modified Ligand Gated Ion Channels

Provided are nucleic acids, polynucleotides, and viral vectors encoding modified ligand gated ion channels (LGICs) that have increased sensitivity towards an exogenous ligand and decreased sensitivity towards an endogenous ligand. In some aspects, the modified LGIC is a chimeric LGIC. In some aspects, the chimeric LGIC comprises a ligand binding domain (LBD) from a first LGIC and an ion pore domain (IPD) from a second LGIC.

In some aspects, a LGIC described herein is a chimeric LGIC comprising a Cys-loop receptor. In some aspects, a LGIC is an acetylcholine receptor (AChR). In some aspects, a LGIC is a neuronal-type AChR (nAChR). In some aspects, an IPD is from a glycine receptor (GlyR).

A modified LGIC subunit described herein can be a modification of an LGIC from any appropriate species (e.g., human, rat, mouse, dog, cat, horse, cow, goat, pig, or monkey). In some aspects, a modified LGIC includes at least one chimeric LGIC subunit having a non-naturally occurring combination of a LBD from a first LGIC and an IPD from a second LGIC.

In some aspects, a modified LGIC (e.g., an LGIC including one or more modified LGIC subunits) can be a homomeric (e.g., having any number of the same modified LGIC subunits) or heteromeric (e.g., having at least one modified LGIC subunit and any number of different LGIC subunits). In some aspects, a modified LGIC can be a homomeric modified LGIC. In some aspects, a modified LGIC described herein can include any suitable number of modified LGIC subunits. In some aspects, a modified LGIC can be a trimer, a tetramer, a pentamer, or a hexamer. In some aspects, a modified LGIC described herein is a pentamer.

In some aspects, where a LGIC includes multiple different subunits (for example, a neuronal-type nAChR includes α4, β2, and α7 subunits), the modified LBD and/or IPD can be selected from any of the subunits.

In some aspects, the chimeric LGIC comprises an LBD from a nicotinic acetylcholine receptor (nAChRs) and an IPD from a glycine receptor (GlyR). A natural nAChR when bound by endogenous ligand activates conductance for cations and typically depolarizes a cell thereby increasing cellular excitability. In contrast, a natural GlyR when bound by endogenous ligand activates chloride anion conductance and typically reduces the excitability of a cell by hyperpolarization.

In some aspects, a chimeric LGIC comprises a nicotinic α7 subtype ACh receptor LBD and a glycine receptor IPD (α7-nAChR-GlyR). In some aspects, when ligand is bound to the α7-nAChR LBD described herein, the chimeric α7-nAChR-GlyR LGIC activates chloride conductance and reduces excitability of a cell by hyperpolarization.

In some aspects, a chimeric LGIC is modified. In some aspects, a α7-nAChR LBD is modified to selectively bind an exogenous ligand. In some aspects, a α7-nAChR LBD is modified to reduce binding to an endogenous ligand.

Various LDB, IPD and constructs are described in Table 1 below.

TABLE 1

| SEQ | | |
|---|---|---|
| ID | | |
| NO. | Description | Sequence |
| 1 | human α7- | MRCSPGGVWLALAASLLHVS |
| | nAChR LBD | LQGEFQRKLYKELVKNYNPL |
| | and GlyR | ERPVANDSQPLTVYFSLSLL |
| | IPD | QIMDVDEKNQVLTTNIWLQM |
| | | SWTDHYLQWNVSEYPGVKTV |
| | | RFPDGQIWKPDILLYNSADE |
| | | RFDATFHTNVGVNSSGHCLY |
| | | LPPGIFKSSCYIDVRWFPFD |
| | | VQHCKLKFGSWSYGGWSLDL |
| | | QMQEADISGYIPNGEWDLVG |
| | | IPGKRSERFYECCKEPFPDV |
| | | TFTVTMRRRMGYYLIQMYIP |
| | | SLLIVILSWISFWINMDAAP |
| | | ARVGLGITTVLTMTTQSSGS |
| | | RASLPKVSYVKAIDIWMAVC |
| | | LLFVFSALLEYAAVNFVSRQ |
| | | HKELLRFRRKRRHHKEDEAG |
| | | EGRFNFSAYGMGPACLQAKD |
| | | GISVKGANNSNTTNPPPAPS |
| | | KSPEEMRKLFIQRAKKIDKI |
| | | SRIGFPMAFLIFNMFYWIIY |
| | | KIVRREDVHNQ |
| 2 | human α7- | ATGCGCTGTTCTCCAGGCGG |
| | nAChR LBD | CGTGTGGCTCGCCCTGGCTG |
| | and GlyR IPD | CTTCCCTTCTGCACGTTAGC |
| | | CTGCAGGGTGAGTTCCAGCG |
| | | CAAACTGTATAAGGAGCTTG |
| | | TTAAGAATTATAACCCCCTG |
| | | GAGCGGCCGGTCGCAAATGA |
| | | TTCCCAGCCACTGACAGTGT |
| | | ACTTCAGCCTCTCCTTGCTG |
| | | CAGATCATGGACGTGGATGA |
| | | AAAGAACCAGGTGCTGACCA |
| | | CTAATATTTGGTTGCAGATG |
| | | TCCTGGACCGATCACTACTT |
| | | GCAGTGGAATGTGAGCGAAT |
| | | ACCCAGGTGTAAAGACTGTA |
| | | AGATTCCCTGACGGCCAAAT |
| | | CTGGAAACCAGATATCCTGC |
| | | TGTACAACAGCGCAGACGAA |
| | | AGGTTTGATGCAACATTTCA |
| | | CACCAACGTGGGAGTCAATT |
| | | CTTCAGGCCACTGCCTGTAC |
| | | CTGCCCCCTGGAATCTTCAA |
| | | GTCCTCATGCTATATCGACG |
| | | TCCGCTGGTTTCCCTTCGAC |
| | | GTCCAGCACTGCAAACTCAA |
| | | ATTCGGGAGCTGGAGCTACG |
| | | GCGGATGGAGCCTGGATCTG |
| | | CAAATGCAGGAGGCTGACAT |
| | | CTCTGGTTACATCCCGAATG |
| | | GGGAGTGGGACCTTGTGGGA |
| | | ATCCCCGGTAAAAGAAGCGA |
| | | GCGATTTTATGAATGCTGCA |
| | | AAGAGCCCTTCCCAGATGTC |
| | | ACCTTCACAGTGACCATGCG |
| | | GAGACGCATGGGTTATTATC |
| | | TGATCCAAATGTATATCCCA |
| | | AGCTTGCTTATAGTGATTTT |
| | | GTCATGGATCTCCTTCTGGA |
| | | TTAATATGGACGCCGCTCCA |
| | | GCTAGGGTCGGACTGGGCAT |
| | | CACCACAGTGCTGACAATGA |
| | | CTACTCAGAGCTCAGGCAGC |
| | | CGAGCCAGCTTGCCCAAGGT |
| | | TTCTTATGTGAAGGCCATCG |
| | | ATATCTGGATGGCTGTCTGC |
| | | CTTCTGTTTGTCTTCAGCGC |
| | | ACTGCTGGAATACGCCGCTG |
| | | TCAATTTTGTGTCTCGACAG |
| | | CATAAAGAGCTGTTGCGGTT |
| | | CAGAAGAAAACGACGCCACC |
| | | ACAAAGAGGATGAGGCAGGA |
| | | GAAGGACGCTTCAACTTTAG |
| | | CGCCTATGGTATGGGACCTG |
| | | CTTGCCTCCAGGCTAAAGAC |
| | | GGAATTTCCGTGAAGGGAGC |
| | | CAACAATAGCAACACAACCA |
| | | ACCCACCCCCTGCTCCATCT |
| | | AAGAGCCCGGAGGAAATGCG |
| | | CAAACTCTTTATTCAGAGAG |
| | | CGAAAAAGATCGACAAAATC |
| | | TCCCGGATCGGATTCCCCAT |
| | | GGCTTTCCTGATTTTCAACA |
| | | TGTTTTATTGGATCATCTAC |
| | | AAGATTGTGCGAAGGGAGGA |
| | | CGTACACAACCAGTAA |
| 3 | rabbit β- | GTGCAGGCTGCCTATCAGAA |
| | globin polyA | GGTGGTGGCTGGTGTGGCCA |
| | | ATGCCCTGGCTCACAAATAC |
| | | CACTGAGATCTTTTTCCCTC |
| | | TGCCAAAAATTATGGGGACA |
| | | TCATGAAGCCCCTTGAGCAT |
| | | CTGACTTCTGGCTAATAAAG |
| | | GAAATTTATTTTCATTGCAA |
| | | TAGTGTGTTGGAATTTTTTG |
| | | TGTCTCTCACTCGGAAGGAC |
| | | ATATGGGAGGGCAAATCATT |
| | | TAAAACATCAGAATGAGTAT |
| | | TTGGTTTAGAGTTTGGCAAC |
| | | ATATGCCCATATGCTGGCTG |
| | | CCATGAACAAAGGTTGGCTA |
| | | TAAAGAGGTCATCAGTATAT |
| | | GAAACAGCCCCCTGCTGTCC |
| | | ATTCCTTATTCCATAGAAAA |
| | | GCCTTGACTTGAGGTTAGAT |
| | | TTTTTTTATATTTTGTTTTG |
| | | TGTTATTTTTTTCTTTAACA |
| | | TCCCTAAAATTTTCCTTACA |
| | | TGTTTTACTAGCCAGATTTT |
| | | TCCTCCTCTCCTGACTACTC |
| | | CCAGTCATAGCTGTCCCTCT |
| | | TCTCTTATGGAGATCCC |
| 4 | human α7- | ATGCGCTGTTCTCCAGGCGG |
| | nAChR LBD- | CGTGTGGCTCGCCCTGGCTG |
| | GlyR IPD- | CTTCCCTTCTGCACGTTAGC |
| | rabbit β | CTGCAGGGTGAGTTCCAGCG |
| | globin polyA | CAAACTGTATAAGGAGCTTG |
| | | TTAAGAATTATAACCCCCTG |
| | | GAGCGGCCGGTCGCAAATGA |
| | | TTCCCAGCCACTGACAGTGT |
| | | ACTTCAGCCTCTCCTTGCTG |
| | | CAGATCATGGACGTGGATGA |
| | | AAAGAACCAGGTGCTGACCA |
| | | CTAATATTTGGTTGCAGATG |
| | | TCCTGGACCGATCACTACTT |
| | | GCAGTGGAATGTGAGCGAAT |
| | | ACCCAGGTGTAAAGACTGTA |
| | | AGATTCCCTGACGGCCAAAT |
| | | CTGGAAACCAGATATCCTGC |
| | | TGTACAACAGCGCAGACGAA |
| | | AGGTTTGATGCAACATTTCA |
| | | CACCAACGTGGGAGTCAATT |
| | | CTTCAGGCCACTGCCTGTAC |
| | | CTGCCCCCTGGAATCTTCAA |
| | | GTCCTCATGCTATATCGACG |
| | | TCCGCTGGTTTCCCTTCGAC |
| | | GTCCAGCACTGCAAACTCAA |
| | | ATTCGGGAGCTGGAGCTACG |
| | | GCGGATGGAGCCTGGATCTG |
| | | CAAATGCAGGAGGCTGACAT |
| | | CTCTGGTTACATCCCGAATG |
| | | GGGAGTGGGACCTTGTGGGA |
| | | ATCCCCGGTAAAAGAAGCGA |
| | | GCGATTTTATGAATGCTGCA |
| | | AAGAGCCCTTCCCAGATGTC |
| | | ACCTTCACAGTGACCATGCG |
| | | GAGACGCATGGGTTATTATC |
| | | TGATCCAAATGTATATCCCA |
| | | AGCTTGCTTATAGTGATTTT |
| | | GTCATGGATCTCCTTCTGGA |
| | | TTAATATGGACGCCGCTCCA |
| | | GCTAGGGTCGGACTGGGCAT |
| | | CACCACAGTGCTGACAATGA |
| | | CTACTCAGAGCTCAGGCAGC |
| | | CGAGCCAGCTTGCCCAAGGT |
| | | TTCTTATGTGAAGGCCATCG |
| | | ATATCTGGATGGCTGTCTGC |
| | | CTTCTGTTTGTCTTCAGCGC |
| | | ACTGCTGGAATACGCCGCTG |
| | | TCAATTTTGTGTCTCGACAG |
| | | CATAAAGAGCTGTTGCGGTT |
| | | CAGAAGAAAACGACGCCACC |
| | | ACAAAGAGGATGAGGCAGGA |
| | | GAAGGACGCTTCAACTTTAG |
| | | CGCCTATGGTATGGGACCTG |
| | | CTTGCCTCCAGGCTAAAGAC |
| | | GGAATTTCCGTGAAGGGAGC |
| | | CAACAATAGCAACACAACCA |
| | | ACCCACCCCCTGCTCCATCT |
| | | AAGAGCCCGGAGGAAATGCG |
| | | CAAACTCTTTATTCAGAGAG |
| | | CGAAAAAGATCGACAAAATC |
| | | TCCCGGATCGGATTCCCCAT |
| | | GGCTTTCCTGATTTTCAACA |
| | | TGTTTTATTGGATCATCTAC |
| | | AAGATTGTGCGAAGGGAGGA |
| | | CGTACACAACCAGTAAGCGG |
| | | CCGCGTGCAGGCTGCCTATC |
| | | AGAAGGTGGTGGCTGGTGTG |
| | | GCCAATGCCCTGGCTCACAA |
| | | ATACCACTGAGATCTTTTTC |
| | | CCTCTGCCAAAAATTATGGG |
| | | GACATCATGAAGCCCCTTGA |
| | | GCATCTGACTTCTGGCTAAT |
| | | AAAGGAAATTTATTTTCATT |
| | | GCAATAGTGTGTTGGAATTT |
| | | TTTGTGTCTCTCACTCGGAA |
| | | GGACATATGGGAGGGCAAAT |
| | | CATTTAAAACATCAGAATGA |
| | | GTATTTGGTTTAGAGTTTGG |
| | | CAACATATGCCCATATGCTG |
| | | GCTGCCATGAACAAAGGTTG |
| | | GCTATAAAGAGGTCATCAGT |
| | | ATATGAAACAGCCCCCTGCT |
| | | GTCCATTCCTTATTCCATAG |
| | | AAAAGCCTTGACTTGAGGTT |
| | | AGATTTTTTTTATATTTTGT |
| | | TTTGTGTTATTTTTTTCTTT |
| | | AACATCCCTAAAATTTTCCT |
| | | TACATGTTTTACTAGCCAGA |
| | | TTTTTCCTCCTCTCCTGACT |
| | | ACTCCCAGTCATAGCTGTCC |
| | | CTCTTCTCTTATGGAGATCC |
| | | C |
| 5 | Chimeric | GTAAGTATCAAGGTTACAAG |
| | intron-Kozak- | ACAGGTTTAAGGAGACCAAT |
| | human α7- | AGAAACTGGGCTTGTCGAGA |
| | nAChR LBD- | CAGAGAAGACTCTTGCGTTT |
| | GlyR IPD- | CTGATAGGCACCTATTGGTC |
| | rabbit β | TTACTGACATCCACTTTGCC |
| | globin polyA | TTTCTCTCCACAGGTGATGA |
| | | TATCACTAGTGCTAGCGCC |
| | | ACCATGCGCTGTTCTCCAGG |
| | | CGGCGTGTGGCTCGCCCTGG |
| | | CTGCTTCCCTTCTGCACGTT |
| | | AGCCTGCAGGGTGAGTTCCA |
| | | GCGCAAACTGTATAAGGAGC |
| | | TTGTTAAGAATTATAACCCC |
| | | CTGGAGCGGCCGGTCGCAAA |
| | | TGATTCCCAGCCACTGACAG |
| | | TGTACTTCAGCCTCTCCTTG |
| | | CTGCAGATCATGGACGTGGA |
| | | TGAAAAGAACCAGGTGCTGA |
| | | CCACTAATATTTGGTTGCAG |
| | | ATGTCCTGGACCGATCACTA |
| | | CTTGCAGTGGAATGTGAGCG |
| | | AATACCCAGGTGTAAAGACT |
| | | GTAAGATTCCCTGACGGCCA |
| | | AATCTGGAAACCAGATATCC |
| | | TGCTGTACAACAGCGCAGAC |
| | | GAAAGGTTTGATGCAACATT |
| | | TCACACCAACGTGGGAGTCA |
| | | ATTCTTCAGGCCACTGCCTG |
| | | TACCTGCCCCCTGGAATCTT |
| | | CAAGTCCTCATGCTATATCG |
| | | ACGTCCGCTGGTTTCCCTTC |
| | | GACGTCCAGCACTGCAAACT |
| | | CAAATTCGGGAGCTGGAGCT |
| | | ACGGCGGATGGAGCCTGGAT |
| | | CTGCAAATGCAGGAGGCTGA |
| | | CATCTCTGGTTACATCCCGA |
| | | ATGGGGAGTGGGACCTTGTG |
| | | GGAATCCCCGGTAAAAGAAG |
| | | CGAGCGATTTTATGAATGCT |
| | | GCAAAGAGCCCTTCCCAGAT |
| | | GTCACCTTCACAGTGACCAT |
| | | GCGGAGACGCATGGGTTATT |
| | | ATCTGATCCAAATGTATATC |
| | | CCAAGCTTGCTTATAGTGAT |
| | | TTTGTCATGGATCTCCTTCT |
| | | GGATTAATATGGACGCCGCT |
| | | CCAGCTAGGGTCGGACTGGG |
| | | CATCACCACAGTGCTGACAA |
| | | TGACTACTCAGAGCTCAGGC |
| | | AGCCGAGCCAGCTTGCCCAA |
| | | GGTTTCTTATGTGAAGGCCA |
| | | TCGATATCTGGATGGCTGTC |
| | | TGCCTTCTGTTTGTCTTCAG |
| | | CGCACTGCTGGAATACGCCG |
| | | CTGTCAATTTTGTGTCTCGA |
| | | CAGCATAAAGAGCTGTTGCG |
| | | GTTCAGAAGAAAACGACGCC |
| | | ACCACAAAGAGGATGAGGCA |
| | | GGAGAAGGACGCTTCAACTT |
| | | TAGCGCCTATGGTATGGGAC |
| | | CTGCTTGCCTCCAGGCTAAA |
| | | GACGGAATTTCCGTGAAGGG |
| | | AGCCAACAATAGCAACACAA |
| | | CCAACCCACCCCCTGCTCCA |
| | | TCTAAGAGCCCGGAGGAAAT |
| | | GCGCAAACTCTTTATTCAGA |
| | | GAGCGAAAAAGATCGACAAA |
| | | ATCTCCCGGATCGGATTCCC |
| | | CATGGCTTTCCTGATTTTCA |
| | | ACATGTTTTATTGGATCATC |
| | | TACAAGATTGTGCGAAGGGA |
| | | GGACGTACACAACCAGTAA |
| | | GCGGCCGCGTGCAGGCTGCC |
| | | TATCAGAAGGTGGTGGCTGG |
| | | TGTGGCCAATGCCCTGGCTC |
| | | ACAAATACCACTGAGATCTT |
| | | TTTCCCTCTGCCAAAAATTA |
| | | TGGGGACATCATGAAGCCCC |
| | | TTGAGCATCTGACTTCTGGC |
| | | TAATAAAGGAAATTTATTTT |
| | | CATTGCAATAGTGTGTTGGA |
| | | ATTTTTTGTGTCTCTCACTC |
| | | GGAAGGACATATGGGAGGGC |
| | | AAATCATTTAAAACATCAGA |
| | | ATGAGTATTTGGTTTAGAGT |
| | | TTGGCAACATATGCCCATAT |
| | | GCTGGCTGCCATGAACAAAG |
| | | GTTGGCTATAAAGAGGTCAT |
| | | CAGTATATGAAACAGCCCCC |
| | | TGCTGTCCATTCCTTATTCC |
| | | ATAGAAAAGCCTTGACTTGA |
| | | GGTTAGATTTTTTTTATATT |
| | | TTGTTTTGTGTTATTTTTTT |
| | | CTTTAACATCCCTAAAATTT |
| | | TCCTTACATGTTTTACTAGC |
| | | CAGATTTTTCCTCCTCTCCT |
| | | GACTACTCCCAGTCATAGCT |
| | | GTCCCTCTTCTCTTATGGAG |
| | | ATCCC |
| 6 | Human | TGACGACCGACCCCGACCCA |
| | synapsin | CTGGACAAGCACCCAACCCC |
| | promoter- | CATTCCCCAAATTGCGCATC |
| | chimeric | CCCTATCAGAGAGGGGGAGG |
| | intron-Kozak- | GGAAACAGGATGCGGCGAGG |
| | human α7- | CGCGTGCGCACTGCCAGCTT |
| | nAChR LBD- | CAGCACCGCGGACAGTGCCT |
| | GlyR IPD- | TCGCCCCCGCCTGGCGGCGC |
| | rabbit β | GCGCCACCGCCGCCTCAGCA |
| | globin polyA | CTGAAGGCGCGCTGACGTCA |
| | | CTCGCCGGTCCCCCGCAAAC |
| | | TCCCCTTCCCGGCCACCTTG |
| | | GTCGCGTCCGCGCCGCCGCC |
| | | GGCCCAGCCGGACCGCACCA |
| | | CGCGAGGCGCGAGATAGGGG |
| | | GGCACGGGCGCGACCATCTG |
| | | CGCTGCGGCGCCGGCGACTC |
| | | AGCGCTGCCTCAGTCTGCGG |
| | | TGGGCAGCGGAGGAGTCGTG |
| | | TCGTGCCTGAGAGCGCAGCT |
| | | GTGCTCCTGGGCACCGCGCA |
| | | GTCCGCCCCCGCGGCTCCTG |
| | | GCCAGACCACCCCTAGGACC |
| | | CCCTGCCCCAAGTCGCAGCC |
| | | GTTGGATCAGGTAAGTATCA |
| | | AGGTTACAAGACAGGTTTAA |
| | | GGAGACCAATAGAAACTGGG |
| | | CTTGTCGAGACAGAGAAGAC |
| | | TCTTGCGTTTCTGATAGGCA |
| | | CCTATTGGTCTTACTGACAT |
| | | CCACTTTGCCTTTCTCTCCA |
| | | CAGGTGATGATATCACTAGT |
| | | GCTAGCGCCACCATGCGCT |
| | | GTTCTCCAGGCGGCGTGTGG |
| | | CTCGCCCTGGCTGCTTCCCT |
| | | TCTGCACGTTAGCCTGCAGG |
| | | GTGAGTTCCAGCGCAAACTG |
| | | TATAAGGAGCTTGTTAAGAA |
| | | TTATAACCCCCTGGAGCGGC |
| | | CGGTCGCAAATGATTCCCAG |
| | | CCACTGACAGTGTACTTCAG |
| | | CCTCTCCTTGCTGCAGATCA |
| | | TGGACGTGGATGAAAAGAAC |
| | | CAGGTGCTGACCACTAATAT |
| | | TTGGTTGCAGATGTCCTGGA |
| | | CCGATCACTACTTGCAGTGG |
| | | AATGTGAGCGAATACCCAGG |
| | | TGTAAAGACTGTAAGATTCC |
| | | CTGACGGCCAAATCTGGAAA |
| | | CCAGATATCCTGCTGTACAA |
| | | CAGCGCAGACGAAAGGTTTG |
| | | ATGCAACATTTCACACCAAC |
| | | GTGGGAGTCAATTCTTCAGG |
| | | CCACTGCCTGTACCTGCCCC |
| | | CTGGAATCTTCAAGTCCTCA |
| | | TGCTATATCGACGTCCGCTG |
| | | GTTTCCCTTCGACGTCCAGC |
| | | ACTGCAAACTCAAATTCGGG |
| | | AGCTGGAGCTACGGCGGATG |
| | | GAGCCTGGATCTGCAAATGC |
| | | AGGAGGCTGACATCTCTGGT |
| | | TACATCCCGAATGGGGAGTG |
| | | GGACCTTGTGGGAATCCCCG |
| | | GTAAAAGAAGCGAGCGATTT |
| | | TATGAATGCTGCAAAGAGCC |
| | | CTTCCCAGATGTCACCTTCA |
| | | CAGTGACCATGCGGAGACGC |
| | | ATGGGTTATTATCTGATCCA |
| | | AATGTATATCCCAAGCTTGC |
| | | TTATAGTGATTTTGTCATGG |
| | | ATCTCCTTCTGGATTAATAT |
| | | GGACGCCGCTCCAGCTAGGG |
| | | TCGGACTGGGCATCACCACA |
| | | GTGCTGACAATGACTACTCA |
| | | GAGCTCAGGCAGCCGAGCCA |
| | | GCTTGCCCAAGGTTTCTTAT |
| | | GTGAAGGCCATCGATATCTG |
| | | GATGGCTGTCTGCCTTCTGT |
| | | TTGTCTTCAGCGCACTGCTG |
| | | GAATACGCCGCTGTCAATTT |
| | | TGTGTCTCGACAGCATAAAG |
| | | AGCTGTTGCGGTTCAGAAGA |
| | | AAACGACGCCACCACAAAGA |
| | | GGATGAGGCAGGAGAAGGAC |
| | | GCTTCAACTTTAGCGCCTAT |
| | | GGTATGGGACCTGCTTGCCT |
| | | CCAGGCTAAAGACGGAATTT |
| | | CCGTGAAGGGAGCCAACAAT |
| | | AGCAACACAACCAACCCACC |
| | | CCCTGCTCCATCTAAGAGCC |
| | | CGGAGGAAATGCGCAAACTC |
| | | TTTATTCAGAGAGCGAAAAA |
| | | GATCGACAAAATCTCCCGGA |
| | | TCGGATTCCCCATGGCTTTC |
| | | CTGATTTTCAACATGTTTTA |
| | | TTGGATCATCTACAAGATTG |
| | | TGCGAAGGGAGGACGTACAC |
| | | AACCAGTAAGCGGCCGCGT |
| | | GCAGGCTGCCTATCAGAAGG |
| | | TGGTGGCTGGTGTGGCCAAT |
| | | GCCCTGGCTCACAAATACCA |
| | | CTGAGATCTTTTTCCCTCTG |
| | | CCAAAAATTATGGGGACATC |
| | | ATGAAGCCCCTTGAGCATCT |
| | | GACTTCTGGCTAATAAAGGA |
| | | AATTTATTTTCATTGCAATA |
| | | GTGTGTTGGAATTTTTTGTG |
| | | TCTCTCACTCGGAAGGACAT |
| | | ATGGGAGGGCAAATCATTTA |
| | | AAACATCAGAATGAGTATTT |
| | | GGTTTAGAGTTTGGCAACAT |
| | | ATGCCCATATGCTGGCTGCC |
| | | ATGAACAAAGGTTGGCTATA |
| | | AAGAGGTCATCAGTATATGA |
| | | AACAGCCCCCTGCTGTCCAT |
| | | TCCTTATTCCATAGAAAAGC |
| | | CTTGACTTGAGGTTAGATTT |
| | | TTTTTATATTTTGTTTTGTG |
| | | TTATTTTTTTCTTTAACATC |
| | | CCTAAAATTTTCCTTACATG |
| | | TTTTACTAGCCAGATTTTTC |
| | | CTCCTCTCCTGACTACTCCC |
| | | AGTCATAGCTGTCCCTCTTC |
| | | TCTTATGGAGATCCC |
| 7 | AAV2 ITR- | CTGCGCGCTCGCTCGCTCAC |
| | human | TGAGGCCGCCCGGGCAAAGC |
| | synapsin | CCGGGCGTCGGGCGACCTTT |
| | promoter- | GGTCGCCCGGCCTCAGTGAG |
| | chimeric | CGAGCGAGCGCGCAGAGAGG |
| | intron-human | GAGTGGCCAACTCCATCACT |
| | α7-nAChR | AGGGGTTCCTTGTAGTTAAT |
| | LBD-GlyR | GATTAACCCGCCATGCTACT |
| | IPD-rabbit β | TATCTACGTAGCCATGCTCT |
| | globin polyA- | AGGAAGATCCGAGCTCCAGT |
| | AAV2-ITR | GTGCTGGAATTCGCCCTTTT |
| | | CAGTATTTAAATTGACGACC |
| | | GACCCCGACCCACTGGACAA |
| | | GCACCCAACCCCCATTCCCC |
| | | AAATTGCGCATCCCCTATCA |
| | | GAGAGGGGGAGGGGAAACAG |
| | | GATGCGGCGAGGCGCGTGCG |
| | | CACTGCCAGCTTCAGCACCG |
| | | CGGACAGTGCCTTCGCCCCC |
| | | GCCTGGCGGCGCGCGCCACC |
| | | GCCGCCTCAGCACTGAAGGC |
| | | GCGCTGACGTCACTCGCCGG |
| | | TCCCCCGCAAACTCCCCTTC |
| | | CCGGCCACCTTGGTCGCGTC |
| | | CGCGCCGCCGCCGGCCCAGC |
| | | CGGACCGCACCACGCGAGGC |
| | | GCGAGATAGGGGGGCACGGG |
| | | CGCGACCATCTGCGCTGCGG |
| | | CGCCGGCGACTCAGCGCTGC |
| | | CTCAGTCTGCGGTGGGCAGC |
| | | GGAGGAGTCGTGTCGTGCCT |
| | | GAGAGCGCAGCTGTGCTCCT |
| | | GGGCACCGCGCAGTCCGCCC |
| | | CCGCGGCTCCTGGCCAGACC |
| | | ACCCCTAGGACCCCCTGCCC |
| | | CAAGTCGCAGCCGTTGGATC |
| | | AGGTAAGTATCAAGGTTACA |
| | | AGACAGGTTTAAGGAGACCA |
| | | ATAGAAACTGGGCTTGTCGA |
| | | GACAGAGAAGACTCTTGCGT |
| | | TTCTGATAGGCACCTATTGG |
| | | TCTTACTGACATCCACTTTG |
| | | CCTTTCTCTCCACAGGTGAT |
| | | GATATCACTAGTGCTAGCGC |
| | | CACCATGCGCTGTTCTCCAG |
| | | GCGGCGTGTGGCTCGCCCTG |
| | | GCTGCTTCCCTTCTGCACGT |
| | | TAGCCTGCAGGGTGAGTTCC |
| | | AGCGCAAACTGTATAAGGAG |
| | | CTTGTTAAGAATTATAACCC |
| | | CCTGGAGCGGCCGGTCGCAA |
| | | ATGATTCCCAGCCACTGACA |
| | | GTGTACTTCAGCCTCTCCTT |
| | | GCTGCAGATCATGGACGTGG |
| | | ATGAAAAGAACCAGGTGCTG |
| | | ACCACTAATATTTGGTTGCA |
| | | GATGTCCTGGACCGATCACT |
| | | ACTTGCAGTGGAATGTGAGC |
| | | GAATACCCAGGTGTAAAGAC |
| | | TGTAAGATTCCCTGACGGCC |
| | | AAATCTGGAAACCAGATATC |
| | | CTGCTGTACAACAGCGCAGA |
| | | CGAAAGGTTTGATGCAACAT |
| | | TTCACACCAACGTGGGAGTC |
| | | AATTCTTCAGGCCACTGCCT |
| | | GTACCTGCCCCCTGGAATCT |
| | | TCAAGTCCTCATGCTATATC |
| | | GACGTCCGCTGGTTTCCCTT |
| | | CGACGTCCAGCACTGCAAAC |
| | | TCAAATTCGGGAGCTGGAGC |
| | | TACGGCGGATGGAGCCTGGA |
| | | TCTGCAAATGCAGGAGGCTG |
| | | ACATCTCTGGTTACATCCCG |
| | | AATGGGGAGTGGGACCTTGT |
| | | GGGAATCCCCGGTAAAAGAA |
| | | GCGAGCGATTTTATGAATGC |
| | | TGCAAAGAGCCCTTCCCAGA |
| | | TGTCACCTTCACAGTGACCA |
| | | TGCGGAGACGCATGGGTTAT |
| | | TATCTGATCCAAATGTATAT |
| | | CCCAAGCTTGCTTATAGTGA |
| | | TTTTGTCATGGATCTCCTTC |
| | | TGGATTAATATGGACGCCGC |
| | | TCCAGCTAGGGTCGGACTGG |
| | | GCATCACCACAGTGCTGACA |
| | | ATGACTACTCAGAGCTCAGG |
| | | CAGCCGAGCCAGCTTGCCCA |
| | | AGGTTTCTTATGTGAAGGCC |
| | | ATCGATATCTGGATGGCTGT |
| | | CTGCCTTCTGTTTGTCTTCA |
| | | GCGCACTGCTGGAATACGCC |
| | | GCTGTCAATTTTGTGTCTCG |
| | | ACAGCATAAAGAGCTGTTGC |
| | | GGTTCAGAAGAAAACGACGC |
| | | CACCACAAAGAGGATGAGGC |
| | | AGGAGAAGGACGCTTCAACT |
| | | TTAGCGCCTATGGTATGGGA |
| | | CCTGCTTGCCTCCAGGCTAA |
| | | AGACGGAATTTCCGTGAAGG |
| | | GAGCCAACAATAGCAACACA |
| | | ACCAACCCACCCCCTGCTCC |
| | | ATCTAAGAGCCCGGAGGAAA |
| | | TGCGCAAACTCTTTATTCAG |
| | | AGAGCGAAAAAGATCGACAA |
| | | AATCTCCCGGATCGGATTCC |
| | | CCATGGCTTTCCTGATTTTC |
| | | AACATGTTTTATTGGATCAT |
| | | CTACAAGATTGTGCGAAGGG |
| | | AGGACGTACACAACCAGTAA |
| | | GCGGCCGCGTGCAGGCTGCC |
| | | TATCAGAAGGTGGTGGCTGG |
| | | TGTGGCCAATGCCCTGGCTC |
| | | ACAAATACCACTGAGATCTT |
| | | TTTCCCTCTGCCAAAAATTA |
| | | TGGGGACATCATGAAGCCCC |
| | | TTGAGCATCTGACTTCTGGC |
| | | TAATAAAGGAAATTTATTTT |
| | | CATTGCAATAGTGTGTTGGA |
| | | ATTTTTTGTGTCTCTCACTC |
| | | GGAAGGACATATGGGAGGGC |
| | | AAATCATTTAAAACATCAGA |
| | | ATGAGTATTTGGTTTAGAGT |
| | | TTGGCAACATATGCCCATAT |
| | | GCTGGCTGCCATGAACAAAG |
| | | GTTGGCTATAAAGAGGTCAT |
| | | CAGTATATGAAACAGCCCCC |
| | | TGCTGTCCATTCCTTATTCC |
| | | ATAGAAAAGCCTTGACTTGA |
| | | GGTTAGATTTTTTTTATATT |
| | | TTGTTTTGTGTTATTTTTTT |
| | | CTTTAACATCCCTAAAATTT |
| | | TCCTTACATGTTTTACTAGC |
| | | CAGATTTTTCCTCCTCTCCT |
| | | GACTACTCCCAGTCATAGCT |
| | | GTCCCTCTTCTCTTATGGAG |
| | | ATCCCGTTAACAACAACAAT |
| | | TGCATTCATTTTATGTTTCA |
| | | GGTTCAGGGGGAGATGTGGG |
| | | AGGTTTTTTAAAGCAAGTAA |
| | | AACCTCTACAAATGTGGTAA |
| | | AATCGATAAGGATCTTCCTA |
| | | GAGCATGGCTACGTAGATAA |
| | | GTAGCATGGCGGGTTAATCA |
| | | TTAACTACAAGGAACCCCTA |
| | | GTGATGGAGTTGGCCACTCC |
| | | CTCTCTGCGCGCTCGCTCGC |
| | | TCACTGAGGCCGGGCGACCA |
| | | AAGGTCGCCCGACGCCCGGG |
| | | CTTTGCCCGGGCGGCCTCAG |
| | | TGAGCGAGCGAGCGCGCAG |
| 8 | AAV2 ITR- | AGCGCCCAATACGCAAACCG |
| | human | CCTCTCCCCGCGCGTTGGCC |
| | synapsin | GATTCATTAATGCAGCTGCG |
| | promoter- | CGCTCGCTCGCTCACTGAGG |
| | chimeric | CCGCCCGGGCAAAGCCCGGG |
| | intron- | CGTCGGGCGACCTTTGGTCG |
| | human | CCCGGCCTCAGTGAGCGAGC |
| | α7-nAChR | GAGCGCGCAGAGAGGGAGTG |
| | LBD-GlyR | GCCAACTCCATCACTAGGGG |
| | IPD-rabbit β | TTCCTTGTAGTTAATGATTA |
| | globin polyA- | ACCCGCCATGCTACTTATCT |
| | AAV2-ITR- | ACGTAGCCATGCTCTAGGAA |
| | kanR-ori | GATCCGAGCTCCAGTGTGCT |
| | plasmid | GGAATTCGCCCTTTTCAGT |
| | | ATTTAAATTGACGACCGACC |
| | | CCGACCCACTGGACAAGCAC |
| | | CCAACCCCCATTCCCCAAAT |
| | | TGCGCATCCCCTATCAGAGA |
| | | GGGGGAGGGGAAACAGGATG |
| | | CGGCGAGGCGCGTGCGCACT |
| | | GCCAGCTTCAGCACCGCGGA |
| | | CAGTGCCTTCGCCCCCGCCT |
| | | GGCGGCGCGCGCCACCGCCG |
| | | CCTCAGCACTGAAGGCGCGC |
| | | TGACGTCACTCGCCGGTCCC |
| | | CCGCAAACTCCCCTTCCCGG |
| | | CCACCTTGGTCGCGTCCGCG |
| | | CCGCCGCCGGCCCAGCCGGA |
| | | CCGCACCACGCGAGGCGCGA |
| | | GATAGGGGGGCACGGGCGCG |
| | | ACCATCTGCGCTGCGGCGCC |
| | | GGCGACTCAGCGCTGCCTCA |
| | | GTCTGCGGTGGGCAGCGGAG |
| | | GAGTCGTGTCGTGCCTGAGA |
| | | GCGCAGCTGTGCTCCTGGGC |
| | | ACCGCGCAGTCCGCCCCCGC |
| | | GGCTCCTGGCCAGACCACCC |
| | | CTAGGACCCCCTGCCCCAAG |
| | | TCGCAGCCGTTGGATCAGGT |
| | | AAGTATCAAGGTTACAAGAC |
| | | AGGTTTAAGGAGACCAATAG |
| | | AAACTGGGCTTGTCGAGACA |
| | | GAGAAGACTCTTGCGTTTCT |
| | | GATAGGCACCTATTGGTCTT |
| | | ACTGACATCCACTTTGCCTT |
| | | TCTCTCCACAGGTGATGATA |
| | | TCACTAGTGCTAGCGCCACC |
| | | ATGCGCTGTTCTCCAGGCGG |
| | | CGTGTGGCTCGCCCTGGCTG |
| | | CTTCCCTTCTGCACGTTAGC |
| | | CTGCAGGGTGAGTTCCAGCG |
| | | CAAACTGTATAAGGAGCTTG |
| | | TTAAGAATTATAACCCCCTG |
| | | GAGCGGCCGGTCGCAAATGA |
| | | TTCCCAGCCACTGACAGTGT |
| | | ACTTCAGCCTCTCCTTGCTG |
| | | CAGATCATGGACGTGGATGA |
| | | AAAGAACCAGGTGCTGACCA |
| | | CTAATATTTGGTTGCAGATG |
| | | TCCTGGACCGATCACTACTT |
| | | GCAGTGGAATGTGAGCGAAT |
| | | ACCCAGGTGTAAAGACTGTA |
| | | AGATTCCCTGACGGCCAAAT |
| | | CTGGAAACCAGATATCCTGC |
| | | TGTACAACAGCGCAGACGAA |
| | | AGGTTTGATGCAACATTTCA |
| | | CACCAACGTGGGAGTCAATT |
| | | CTTCAGGCCACTGCCTGTAC |
| | | CTGCCCCCTGGAATCTTCAA |
| | | GTCCTCATGCTATATCGACG |
| | | TCCGCTGGTTTCCCTTCGAC |
| | | GTCCAGCACTGCAAACTCAA |
| | | ATTCGGGAGCTGGAGCTACG |
| | | GCGGATGGAGCCTGGATCTG |
| | | CAAATGCAGGAGGCTGACAT |
| | | CTCTGGTTACATCCCGAATG |
| | | GGGAGTGGGACCTTGTGGGA |
| | | ATCCCCGGTAAAAGAAGCGA |
| | | GCGATTTTATGAATGCTGCA |
| | | AAGAGCCCTTCCCAGATGTC |
| | | ACCTTCACAGTGACCATGCG |
| | | GAGACGCATGGGTTATTATC |
| | | TGATCCAAATGTATATCCCA |
| | | AGCTTGCTTATAGTGATTTT |
| | | GTCATGGATCTCCTTCTGGA |
| | | TTAATATGGACGCCGCTCCA |
| | | GCTAGGGTCGGACTGGGCAT |
| | | CACCACAGTGCTGACAATGA |
| | | CTACTCAGAGCTCAGGCAGC |
| | | CGAGCCAGCTTGCCCAAGGT |
| | | TTCTTATGTGAAGGCCATCG |
| | | ATATCTGGATGGCTGTCTGC |
| | | CTTCTGTTTGTCTTCAGCGC |
| | | ACTGCTGGAATACGCCGCTG |
| | | TCAATTTTGTGTCTCGACAG |
| | | CATAAAGAGCTGTTGCGGTT |
| | | CAGAAGAAAACGACGCCACC |
| | | ACAAAGAGGATGAGGCAGGA |
| | | GAAGGACGCTTCAACTTTAG |
| | | CGCCTATGGTATGGGACCTG |
| | | CTTGCCTCCAGGCTAAAGAC |
| | | GGAATTTCCGTGAAGGGAGC |
| | | CAACAATAGCAACACAACCA |
| | | ACCCACCCCCTGCTCCATCT |
| | | AAGAGCCCGGAGGAAATGCG |
| | | CAAACTCTTTATTCAGAGAG |
| | | CGAAAAAGATCGACAAAATC |
| | | TCCCGGATCGGATTCCCCAT |
| | | GGCTTTCCTGATTTTCAACA |
| | | TGTTTTATTGGATCATCTAC |
| | | AAGATTGTGCGAAGGGAGGA |
| | | CGTACACAACCAGTAAGCGG |
| | | CCGCGTGCAGGCTGCCTATC |
| | | AGAAGGTGGTGGCTGGTGTG |
| | | GCCAATGCCCTGGCTCACAA |
| | | ATACCACTGAGATCTTTTTC |
| | | CCTCTGCCAAAAATTATGGG |
| | | GACATCATGAAGCCCCTTGA |
| | | GCATCTGACTTCTGGCTAAT |
| | | AAAGGAAATTTATTTTCATT |
| | | GCAATAGTGTGTTGGAATTT |
| | | TTTGTGTCTCTCACTCGGAA |
| | | GGACATATGGGAGGGCAAAT |
| | | CATTTAAAACATCAGAATGA |
| | | GTATTTGGTTTAGAGTTTGG |
| | | CAACATATGCCCATATGCTG |
| | | GCTGCCATGAACAAAGGTTG |
| | | GCTATAAAGAGGTCATCAGT |
| | | ATATGAAACAGCCCCCTGCT |
| | | GTCCATTCCTTATTCCATAG |
| | | AAAAGCCTTGACTTGAGGTT |
| | | AGATTTTTTTTATATTTTGT |
| | | TTTGTGTTATTTTTTTCTTT |
| | | AACATCCCTAAAATTTTCCT |
| | | TACATGTTTTACTAGCCAGA |
| | | TTTTTCCTCCTCTCCTGACT |
| | | ACTCCCAGTCATAGCTGTCC |
| | | CTCTTCTCTTATGGAGATCC |
| | | CGTTAACAACAACAATTGCA |
| | | TTCATTTTATGTTTCAGGTT |
| | | CAGGGGGAGATGTGGGAGGT |
| | | TTTTTAAAGCAAGTAAAACC |
| | | TCTACAAATGTGGTAAAATC |
| | | GATAAGGATCTTCCTAGAGC |
| | | ATGGCTACGTAGATAAGTAG |
| | | CATGGGGGTTAATCATTAAC |
| | | TACAAGGAACCCCTAGTGAT |
| | | GGAGTTGGCCACTCCCTCTC |
| | | TGCGCGCTCGCTCGCTCACT |
| | | GAGGCCGGGCGACCAAAGGT |
| | | CGCCCGACGCCCGGGCTTTG |
| | | CCCGGGCGGCCTCAGTGAGC |
| | | GAGCGAGCGCGCAGCTGGCG |
| | | TAATAGCGAAGAGGCCCGCA |
| | | CCGATCGCCCTTCCCAACAG |
| | | TTGCGCAGCCTGAATGGCGA |
| | | ATGGGACGCGCCCTGTAGCG |
| | | GCGCATTAAGCGCGGCGGGT |
| | | GTGGTGGTTACGCGCAGCGT |
| | | GACCGCTACACTTGCCAGCG |
| | | CCCTAGCGCCCGCTCCTTTC |
| | | GCTTTCTTCCCTTCCTTTCT |
| | | CGCCACGTTCGCCGGCTTTC |
| | | CCCGTCAAGCTCTAAATCGG |
| | | GGGCTCCCTTTAGGGTTCCG |
| | | ATTTAGTGCTTTACGGCACC |
| | | TCGACCCCAAAAAACTTGAT |
| | | TAGGGTGATGGTTCACGTAG |
| | | TGGGCCATCGCCCTGATAGA |
| | | CGGTTTTTCGCCCTTTGACG |
| | | TTGGAGTCCACGTTCTTTAA |
| | | TAGTGGACTCTTGTTCCAAA |
| | | CTGGAACAACACTCAACCCT |
| | | ATCTCGGTCTATTCTTTTGA |
| | | TTTATAAGGGATTTTGCCGA |
| | | TTTCGGCCTATTGGTTAAAA |
| | | AATGAGCTGATTTAACAAAA |
| | | ATTTAACGCGAATTTTAACA |
| | | AAATATTAACGCTTACAATT |
| | | TAGGTGGCACTTTTCGGGGA |
| | | AATGTGCGCGGAACCCCTAT |
| | | TTGTTTATTTTTCTAAATAC |
| | | ATTCAAATATGTATCCGCTC |
| | | ATGAGACAATAACCCTGATA |
| | | AATGCTTCAATAATATTGAA |
| | | AAAGGAAGAGTATGATTGAA |
| | | CAAGATGGATTGCACGCAGG |
| | | TTCTCCGGCCGCTTGGGTGG |
| | | AGAGGCTATTCGGCTATGAC |
| | | TGGGCACAACAGACAATCGG |
| | | CTGCTCTGATGCCGCCGTGT |
| | | TCCGGCTGTCAGCGCAGGGG |
| | | CGCCCGGTTCTTTTTGTCAA |
| | | GACCGACCTGTCCGGTGCCC |
| | | TGAATGAACTGCAAGACGAG |
| | | GCAGCGCGGCTATCGTGGCT |
| | | GGCCACGACGGGCGTTCCTT |
| | | GCGCAGCTGTGCTCGACGTT |
| | | GTCACTGAAGCGGGAAGGGA |
| | | CTGGCTGCTATTGGGCGAAG |
| | | TGCCGGGGCAGGATCTCCTG |
| | | TCATCTCACCTTGCTCCTGC |
| | | CGAGAAAGTATCCATCATGG |
| | | CTGATGCAATGCGGCGGCTG |
| | | CATACGCTTGATCCGGCTAC |
| | | CTGCCCATTCGACCACCAAG |
| | | CGAAACATCGCATCGAGCGA |
| | | GCACGTACTCGGATGGAAGC |
| | | CGGTCTTGTCGATCAGGATG |
| | | ATCTGGACGAAGAGCATCAG |
| | | GGGCTCGCGCCAGCCGAACT |
| | | GTTCGCCAGGCTCAAGGCGA |
| | | GCATGCCCGACGGCGAGGAT |
| | | CTCGTCGTGACCCATGGCGA |
| | | TGCCTGCTTGCCGAATATCA |
| | | TGGTGGAAAATGGCCGCTTT |
| | | TCTGGATTCATCGACTGTGG |
| | | CCGGCTGGGTGTGGCGGACC |
| | | GCTATCAGGACATAGCGTTG |
| | | GCTACCCGTGATATTGCTGA |
| | | AGAGCTTGGCGGCGAATGGG |
| | | CTGACCGCTTCCTCGTGCTT |
| | | TACGGTATCGCCGCTCCCGA |
| | | TTCGCAGCGCATCGCCTTCT |
| | | ATCGCCTTCTTGACGAGTTC |
| | | TTCTGACTGTCAGACCAAGT |
| | | TTACTCATATATACTTTAGA |
| | | TTGATTTAAAACTTCATTTT |
| | | TAATTTAAAAGGATCTAGGT |
| | | GAAGATCCTTTTTGATAATC |
| | | TCATGACCAAAATCCCTTAA |
| | | CGTGAGTTTTCGTTCCACTG |
| | | AGCGTCAGACCCCGTAGAAA |
| | | AGATCAAAGGATCTTCTTGA |
| | | GATCCTTTTTTTCTGCGCGT |
| | | AATCTGCTGCTTGCAAACAA |
| | | AAAAACCACCGCTACCAGCG |
| | | GTGGTTTGTTTGCCGGATCA |
| | | AGAGCTACCAACTCTTTTTC |
| | | CGAAGGTAACTGGCTTCAGC |
| | | AGAGCGCAGATACCAAATAC |
| | | TGTTCTTCTAGTGTAGCCGT |
| | | AGTTAGGCCACCACTTCAAG |
| | | AACTCTGTAGCACCGCCTAC |
| | | ATACCTCGCTCTGCTAATCC |
| | | TGTTACCAGTGGCTGCTGCC |
| | | AGTGGCGATAAGTCGTGTCT |
| | | TACCGGGTTGGACTCAAGAC |
| | | GATAGTTACCGGATAAGGCG |
| | | CAGCGGTCGGGCTGAACGGG |
| | | GGGTTCGTGCACACAGCCCA |
| | | GCTTGGAGCGAACGACCTAC |
| | | ACCGAACTGAGATACCTACA |
| | | GCGTGAGCTATGAGAAAGCG |
| | | CCACGCTTCCCGAAGGGAGA |
| | | AAGGCGGACAGGTATCCGGT |
| | | AAGCGGCAGGGTCGGAACAG |
| | | GAGAGCGCACGAGGGAGCTT |
| | | CCAGGGGGAAACGCCTGGTA |
| | | TCTTTATAGTCCTGTCGGGT |
| | | TTCGCCACCTCTGACTTGAG |
| | | CGTCGATTTTTGTGATGCTC |
| | | GTCAGGGGGGCGGAGCCTAT |
| | | GGAAAAACGCCAGCAACGCG |
| | | GCCTTTTTACGGTTCCTGGC |
| | | CTTTTGCTGGCCTTTTGCTC |
| | | ACATGTTCTTTCCTGCGTTA |
| | | TCCCCTGATTCTGTGGATAA |
| | | CCGTATTACCGCCTTTGAGT |
| | | GAGCTGATACCGCTCGCCGC |
| | | AGCCGAACGACCGAGCGCAG |
| | | CGAGTCAGTGAGCGAGGAAG |
| | | CGGAAG |
| 9 | AAV5 VP1 | MSFVDHPPDW LEEVGEGLRE |
| | NCBI | FLGLEAGPPK PKPNQQHQDQ |
| | YP_068409.1 | ARGLVLPGYN YLGPGNGLDR |
| | | GEPVNRADEV AREHDISYNE |
| | | QLEAGDNPYL KYNHADAEFQ |
| | | EKLADDTSFG GNLGKAVFQA |
| | | KKRVLEPFGL VEEGAKTAPT |
| | | GKRIDDHFPK RKKARTEEDS |
| | | KPSTSSDAEA GPSGSQQLQI |
| | | PAQPASSLGA DTMSAGGGGP |
| | | LGDNNQGADG VGNASGDWHC |
| | | DSTWMGDRVV TKSTRTWVLP |
| | | SYNNHQYREI KSGSVDGSNA |
| | | NAYFGYSTPW GYFDFNRFHS |
| | | HWSPRDWQRL INNYWGFRPR |
| | | SLRVKIFNIQ VKEVTVQDST |
| | | TTIANNLTST VQVFTDDDYQ |
| | | LPYVVGNGTE GCLPAFPPQV |
| | | FTLPQYGYAT LNRDNTENPT |
| | | ERSSFFCLEY FPSKMLRTGN |
| | | NFEFTYNFEE VPFHSSFAPS |
| | | QNLFKLANPL VDQYLYRFVS |
| | | TNNTGGVQFN KNLAGRYANT |
| | | YKNWFPGPMG RTQGWNLGSG |
| | | VNRASVSAFA TTNRMELEGA |
| | | SYQVPPQPNG MTNNLQGSNT |
| | | YALENTMIFN SQPANPGTTA |
| | | TYLEGNMLIT SESETQPVNR |
| | | VAYNVGGQMA TNNQSSTTAP |
| | | ATGTYNLQEI VPGSVWMERD |
| | | VYLQGPIWAK IPETGAHFHP |
| | | SPAMGGFGLK HPPPMMLIKN |
| | | TPVPGNITSF SDVPVSSFIT |
| | | QYSTGQVTVE MEWELKKENS |
| | | KRWNPEIQYT NNYNDPQFVD |
| | | FAPDSTGEYR TTRPIGTRYL |
| | | TRPL |
| 10 | 5′ ITR | CTGCGCGCTCGCTCGCTCAC |
| | | TGAGGCCGCCCGGGCAAAGC |
| | | CCGGGCGTCGGGCGACCTTT |
| | | GGTCGCCCGGCCTCAGTGAG |
| | | CGAGCGAGCGCGCAGAGAGG |
| | | GAGTGGCCAACTCCATCACT |
| | | AGGGGTTCCT |
| 11 | Human | TGACGACCGACCCCGACCCA |
| | synapsin | CTGGACAAGCACCCAACCCC |
| | promoter | CATTCCCCAAATTGCGCATC |
| | | CCCTATCAGAGAGGGGGAGG |
| | | GGAAACAGGATGCGGCGAGG |
| | | CGCGTGCGCACTGCCAGCTT |
| | | CAGCACCGCGGACAGTGCCT |
| | | TCGCCCCCGCCTGGCGGCGC |
| | | GCGCCACCGCCGCCTCAGCA |
| | | CTGAAGGCGCGCTGACGTCA |
| | | CTCGCCGGTCCCCCGCAAAC |
| | | TCCCCTTCCCGGCCACCTTG |
| | | GTCGCGTCCGCGCCGCCGCC |
| | | GGCCCAGCCGGACCGCACCA |
| | | CGCGAGGCGCGAGATAGGGG |
| | | GGCACGGGCGCGACCATCTG |
| | | CGCTGCGGCGCCGGCGACTC |
| | | AGCGCTGCCTCAGTCTGCGG |
| | | TGGGCAGCGGAGGAGTCGTG |
| | | TCGTGCCTGAGAGCGCAGCT |
| | | GTGCTCCTGGGCACCGCGCA |
| | | GTCCGCCCCCGCGGCTCCTG |
| | | GCCAGACCACCCCTAGGACC |
| | | CCCTGCCCCAAGTCGCAGCC |
| 12 | Intron | GTAAGTATCAAGGTTACAAG |
| | | ACAGGTTTAAGGAGACCAAT |
| | | AGAAACTGGGCTTGTCGAGA |
| | | CAGAGAAGACTCTTGCGTTT |
| | | CTGATAGGCACCTATTGGTC |
| | | TTACTGACATCCACTTTGCC |
| | | TTTCTCTCCACAG |
| | Kozak | GCCACC |
| 13 | 3′ ITR | AGGAACCCCTAGTGATGGAG |
| | | TTGGCCACTCCCTCTCTGCG |
| | | CGCTCGCTCGCTCACTGAGG |
| | | CCGGGCGACCAAAGGTCGCC |
| | | CGACGCCCGGGCTTTGCCCG |
| | | GGCGGCCTCAGTGAGCGAGC |
| | | GAGCGCGCAG |

In some aspects, a modified LGIC subunit or chimeric modified LGIC includes at least one modified amino acid (e.g., an amino acid substitution) in the LBD and/or at least one modified amino acid (e.g., an amino acid substitution) in the IPD.

In some aspects, a modified LGIC or chimeric modified LGIC includes 1-100 amino acid substitutions. In some aspects, a modified LGIC or chimeric modified LGIC comprises 1-10, 11-20, 21-30, 31-40, 41-50, 51-60, 61-70, 71-80, 81-90, or 91-100 amino acid substitutions in SEQ ID NO: 1 or SEQ ID NO: 7.

In some aspects, a modified LGIC subunit or chimeric modified LGIC includes at least one modified nucleic acid in the LBD and/or at least one modified nucleic acid in the IPD.

In some aspects, a modified LGIC subunit or chimeric modified LGIC includes 1-300 nucleic acid substitutions. In some aspects, a modified LGIC subunit or chimeric modified LGIC comprises 1-10, 11-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-240, 241-250, 251-260, 261-270, 271-280, 281-290, or 291-300 nucleic acid substitutions in SEQ ID NO: 1 or SEQ ID NO: 7.

In some aspects, a chimeric modified LGIC has at least 85%, 86%, 87%, 88%, 89%, 90%, 91%, 92%, 93%, or 94% sequence identity to a sequence set forth in SEQ ID NO: 4.

In some aspects, an expression construct comprising a chimeric modified LGIC has at least 85%, 86%, 87%, 88%, 89%, 90%, 91%, 92%, 93%, 94%, 95%, 96%, 97%, 98% or 99% sequence identity to a sequence set forth in SEQ ID NO: 5.

In some aspects, an expression construct comprising a chimeric modified LGIC has at least 85%, 86%, 87%, 88%, 89%, 90%, 91%, 92%, 93%, 94%, 95%, 96%, 97%, 98% or 99% sequence identity to a sequence set forth in SEQ ID NO: 6.

In some aspects, an expression construct comprising a chimeric modified LGIC has at least 85%, 86%, 87%, 88%, 89%, 90%, 91%, 92%, 93%, 94%, 95%, 96%, 97%, 98% or 99% sequence identity to a sequence set forth in SEQ ID NO: 7.

In some aspects, an expression construct comprising a chimeric modified LGIC has at least 90% sequence identity to a sequence set forth in SEQ ID NO: 4, or SEQ ID NO: 5, SEQ ID NO: 6, or SEQ ID NO: 7.

In some aspects, an expression construct comprising a chimeric modified LGIC has at least 95% sequence identity to a sequence set forth in SEQ ID NO: 4, or SEQ ID NO: 5, SEQ ID NO: 6, or SEQ ID NO: 7.

In some aspects, an expression construct comprising a chimeric modified LGIC has at least 96% sequence identity to a sequence set forth in SEQ ID NO: 4, or SEQ ID NO: 5, SEQ ID NO: 6, or SEQ ID NO: 7.

In some aspects, an expression construct comprising a chimeric modified LGIC has at least 97% sequence identity to a sequence set forth in SEQ ID NO: 4, or SEQ ID NO: 5, SEQ ID NO: 6, or SEQ ID NO: 7.

In some aspects, an expression construct comprising a chimeric modified LGIC has at least 98% sequence identity to a sequence set forth in SEQ ID NO: 4, or SEQ ID NO: 5, SEQ ID NO: 6, or SEQ ID NO: 7.

In some aspects, an expression construct comprising a chimeric modified LGIC has at least 99% sequence identity to a sequence set forth in SEQ ID NO: 4, or SEQ ID NO: 5, SEQ ID NO: 6, or SEQ ID NO: 7.

In some aspects, an expression construct comprising a chimeric modified LGIC has a sequence set forth in SEQ ID NO: 4, or SEQ ID NO: 5, SEQ ID NO: 6, or SEQ ID NO: 7.

In some aspects, a chimeric modified LGIC comprises a LBD that comprises an amino acid substitution at amino acid residue 131 as numbered in SEQ ID NO: 1, SEQ ID NO: 3, and SEQ ID NO: 5, wherein the amino acid substitution at residue 131 is L131G.

In some aspects, a chimeric modified LGIC comprises a LBD that comprises an amino acid substitution at amino acid residue 139 as numbered in SEQ ID NO: 1, and SEQ ID NO: 4, wherein the amino acid substitution at residue 139 is Q139L.

In some aspects, a chimeric modified LGIC comprises a LBD that comprises an amino acid substitution at amino acid residue 217 as numbered in SEQ ID NO: 1 and SEQ ID NO: 4, wherein the amino acid substitution at residue 217 is Y217F.

In some aspects, a chimeric modified LGIC comprises a LBD that has an amino acid modification comprising from 1-3 amino acid substitutions at one or more of amino acid residues selected from the group consisting of 131, 139, and 217 as numbered in SEQ ID NO: 1 and SEQ ID NO: 4.

In some aspects, a chimeric modified LGIC comprises two amino acid substitutions at residue 131 and 139 as numbered in SEQ ID NO: 1 and SEQ ID NO: 4, wherein the amino acid substitution at residue 131 is L131G and the amino acid substitution at 139 is Q139L.

In some aspects, a chimeric modified LGIC comprises two amino acid substitutions at residue 131 and 217 as numbered in SEQ ID NO: 1 and SEQ ID NO: 4, wherein the amino acid substitution at residue 131 is L131G and the amino acid substitution at 217 is Y217F.

In some aspects, a chimeric modified LGIC comprises two amino acid substitutions at residue 139 and 217 as numbered in SEQ ID NO: 1 and SEQ ID NO: 4, wherein the amino acid substitution at residue 139 is Q139L and the amino acid substitution at 217 is Y217F.

In some aspects, the LBD and/or IPD is a homolog, orthologue, or paralog of a sequence set forth in SEQ ID NOs: 1-7, and reference to a particular modified amino acid residue can shift to the corresponding homolog, ortholog, or paralog.

In some aspects, a chimeric LGIC is modified such that the α7-nAChR LBD selectively binds varenicline. In some aspects, the modification comprises an amino acid substitution in the α7-nAChR LBD that is selected from L131G, Q139L, and Y217F. In some aspects, the modification in the α7-nAChR LBD comprises L131G, Q139L, and Y217F (α^{L131G, Q139L, Y217F}-GlyR).

In some aspects, the modified LGICs described herein can be used in a method for treating a channelopathy (e.g., a neural channelopathy or a muscle channelopathy). In some aspects, a modified LGIC and an exogenous LGIC ligand that can bind to and activate the modified LGIC are used to treat a subject having a channelopathy. In some aspects, a m-α7-GlyR LGIC and varenicline are used to modulate (e.g., inhibit) ion transport across a membrane of a cell of a mammal. In some aspects, an m-α7-GlyR LGIC and varenicline are used to modulate (e.g., decrease) the excitability of a cell in a mammal.

In some aspects, the modified LGICs described herein can be used for treating neuralgias associated with the trigeminal nerve, more specifically neuralgias caused by hyperexcitability of the trigeminal nerve, including trigeminal neuralgia; trigeminal autonomic cephalgias (“TACs”) including short-lasting unilateral neuralgiform headache attacks with conjunctival injection and tearing (“SUNCT”), short-lasting unilateral neuralgiform headache attacks with cranial autonomic symptoms (“SUNA”), episodic cluster headache, and chronic cluster headache; trigeminal deafferation pain; burning mouth syndrome; or post-traumatic trigeminal neuropathic pain.

In some aspects, a m-α7-GlyR LGIC when administered to a mammal does not have constitutive activity in the mammal in the absence of varenicline.

In some aspects, an m-α7-GlyR LGIC when administered to a mammal does not show tachyphylaxis in the mammal upon sustained treatment of the mammal with varenicline.

In some aspects, the α7^{L131G, Q139L, Y217F}-GlyR LGIC has increased sensitivity (approximately 400-fold) to varenicline relative to the unmodified chimeric α7-GlyR LGIC. In some aspects, varenicline has an EC₅₀ for α7^{L131G, Q139L, Y217}F-GlyR LGIC of 1.6±0.1 nM. In some aspects, the three amino acid modifications in α7^{L131G, Q139L, Y217F}-GlyR LGIC result in decreased sensitivity to acetylcholine while maintaining low potencies to choline and nicotine (>50 μM). In some aspects, varenicline has 160-fold agonist selectivity at α7^{L131G, Q139L, Y217F}-GlyR LGIC over α4β22 nAChR. In some aspects, varenicline is 875-fold more potent at α7^{L131G, Q139L, Y217F}-GlyR LGIC than at 5-Hydroxytryptamine 3 receptor (5HT3R).

In some aspects, m-α7-GlyR LGIC is used in a viral vector for targeted treatment of trigeminal neuralgia in combination with varenicline.

### III. AAV Vectors

A large number of vectors are known and used in the art including, for example, plasmids, modified eukaryotic viruses, or modified bacterial viruses. In some aspects, insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, a delivery vector is selected from the group consisting of a viral vector (e.g., an AAV vector), a plasmid, a lipid, a protein particle, a bacterial vector, and a lysosome.

Adeno-associated viruses (AAVs) have been used as highly effective vehicles capable of carrying specific DNA sequences to target tissues/cells, subsequently allowing to replace, edit or silence faulty genes in many genetic diseases. A number of pre-clinical studies in a range of animal species (mice, rats, pigs, dogs, non-human primates), as well as completed and ongoing clinical trials, have demonstrated safe and effective use of AAVs (Kuzmin et al. Nat Rev Drug Discov 20 (3): 173-174, 2021). While the originally engineered AAV2 serotype remains the most widely used in clinical trials, other capsids have been explored.

In some aspects, the chimeric LGIC is delivered in an “adeno-associated virus” (AAV) vector. In some aspects, the AAV vector can be derived from various serotypes, including combinations of serotypes (i.e., “pseudotyped” AAV) or from various genomes (e.g., single stranded or self-complementary). In addition, the AAV vector can be replication defective and/or targeted. In some aspects, the AAV, includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, bovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. In some aspects, the AAV vector is modified or mutated relative to the wild-type AAV serotype sequence. In some aspects, the AAV vector is an AAV5 vector comprising an AAV5 capsid encapsulating a vector genome comprising AAV2 inverted terminal repeats (ITRs).

### Method for Producing Recombinant AAVs

The present disclosure also provides methods for the generation of AAV particles, by viral genome replication in a viral replication cell comprising contacting the viral replication cell with an AAV polynucleotide or AAV genome (e.g., an AAV vector of the present disclosure). In the context of the present disclosure, the AAV vectors disclosed herein are considered AAV payload construct vectors.

In some aspects, an AAV particle is produced by a method comprising the steps of: (1) co-transfecting competent bacterial cells with a bacmid vector and either a viral construct vector and/or AAV payload construct vector, (2) isolating the resultant viral construct expression vector and AAV payload construct expression vector and separately transfecting viral replication cells, (3) isolating and purifying resultant payload and viral construct particles comprising viral construct expression vector or AAV payload construct expression vector, (4) co-infecting a viral replication cell with both the AAV payload and viral construct particles comprising viral construct expression vector or AAV payload construct expression vector, and (5) harvesting and purifying the viral particle comprising a parvoviral genome.

In some aspects, the present disclosure provides a method for producing an AAV particle comprising the steps of (1) simultaneously co-transfecting mammalian cells, such as, but not limited to HEK293 cells, with a payload region (e.g., polynucleotide encoding a therapeutic protein of the disclosure), a construct expressing rep and cap genes and a helper construct, and (2) harvesting and purifying the AAV particle comprising a viral genome.

In some aspects, the AAV particles can be produced in a viral replication cell that comprises an insect cell. Growing conditions for insect cells in culture, and production of heterologous products in insect cells in culture are well-known in the art, see, e.g., U.S. Pat. No. 6,204,059.

The viral replication cell can be selected from any biological organism, including prokaryotic (e.g., bacterial) cells, and eukaryotic cells, including, insect cells, yeast cells and mammalian cells. Viral replication cells can comprise mammalian cells such as A549, WEH1, 3T3, 10T1/2, BHK, MDCK, COS 1, COS 7, BSC 1, BSC 40, BMT 10, VERO. W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

Viral production disclosed herein describes processes and methods for producing AAV particles that contact a target cell to deliver a payload, e.g. a recombinant viral construct, which comprises a polynucleotide sequence encoding a payload such as a modified chimeric LGIC disclosed herein.

In some aspects, the AAV particles can be produced in a viral replication cell that comprises a mammalian cell. Viral replication cells commonly used for production of recombinant AAV particles include, but are not limited to 293 cells, COS cells, HeLa cells, and KB cells.

In some aspects, AAV particles are produced in mammalian cells wherein all three VP proteins are expressed at a stoichiometry approaching 1:1:10 (VP1: VP2: VP3). The regulatory mechanisms that allow this controlled level of expression include the production of two mRNAs, one for VP1, and the other for VP2 and VP3, produced by differential splicing.

In some aspects, AAV particles are produced in mammalian cells using a triple transfection method wherein a payload construct, parvoviral Rep and parvoviral Cap and a helper construct are comprised within three different constructs. The triple transfection method of the three components of AAV particle production can be utilized to produce small lots of virus for assays including transduction efficiency, target tissue (tropism) evaluation, and stability.

In some aspects, the viral construct vector and the AAV payload construct vector can be each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses can be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see, e.g., Urabe, M. et al., J Virol. 2006 February; 80 (4): 1874-85, the contents of which are herein incorporated by reference in their entirety.

Production of AAV particles with baculovirus in an insect cell system can address known baculovirus genetic and physical instability. Baculovirus-infected viral producing cells are harvested into aliquots that can be cryopreserved in liquid nitrogen; the aliquots retain viability and infectivity for infection of large-scale viral producing cell culture (Wasilko D J et al., Protein Expr Purif. 2009 June; 65 (2): 122-32).

In some aspects, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some aspects, AAV particle production can be modified to increase the scale of production. Transfection of replication cells in large-scale culture formats can be carried out according to any methods known in the art.

In some aspects, cell culture bioreactors can be used for large scale viral production. In some cases, bioreactors comprise stirred tank reactors.

### Cell Lysis

Cells of the disclosure, including, but not limited to viral production cells, can be subjected to cell lysis according to any methods known in the art. Cell lysis can be carried out to obtain one or more agents (e.g. viral particles) present within any cells of the disclosure.

Cell lysis methods can be chemical or mechanical. Chemical cell lysis typically comprises contacting one or more cells with one or more lysis agent. Mechanical lysis typically comprises subjecting one or more cells to one or more lysis condition and/or one or more lysis force. In some aspects, chemical lysis can be used to lyse cells. As used herein, the term “lysis agent” refers to any agent that can aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions can include one or more buffering agents, solubilizing agents, surfactants, preservatives, cryoprotectants, enzymes, enzyme inhibitors and/or chelators.

Concentrations of salts can be increased or decreased to obtain an effective concentration for rupture of cell membranes. Lysis agents comprising detergents can include ionic detergents or non-ionic detergents. Detergents can function to break apart or dissolve cell structures including, but not limited to cell membranes, cell walls, lipids, carbohydrates, lipoproteins and glycoproteins.

In some aspects, mechanical cell lysis is carried out. Mechanical cell lysis methods can include the use of one or more lysis condition and/or one or more lysis force. As used herein, the term “lysis condition” refers to a state or circumstance that promotes cellular disruption. Lysis conditions can comprise certain temperatures, pressures, osmotic purity, salinity and the like. In some aspects, lysis conditions comprise increased or decreased temperatures. In some aspects, lysis conditions comprise changes in temperature to promote cellular disruption. Cell lysis carried out according to such aspects can include freeze-thaw lysis.

As used herein, the term “lysis force” refers to a physical activity used to disrupt a cell. Lysis forces can include, but are not limited to mechanical forces, sonic forces, gravitational forces, optical forces, electrical forces and the like. Cell lysis carried out by mechanical force is referred to herein as “mechanical lysis.” Mechanical forces that can be used according to mechanical lysis can include high shear fluid forces.

In some aspects, a method for harvesting AAV particles without lysis can be used for efficient and scalable AAV particle production. In a non-limiting example, AAV particles can be produced by culturing an AAV particle lacking a heparin binding site, thereby allowing the AAV particle to pass into the supernatant, in a cell culture, collecting supernatant from the culture; and isolating the AAV particle from the supernatant, as described in U.S. Patent Application 20090275107.

### AAV Purification

Cell lysates comprising viral particles can be subjected to clarification. Clarification refers to initial steps taken in purification of viral particles from cell lysates. Clarification serves to prepare lysates for further purification by removing larger, insoluble debris. Clarification steps can include, but are not limited to centrifugation and filtration.

In some aspects, AAV particles can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography), immunoaffinity chromatography and size-exclusion chromatography.

In some aspects, a cell is contacted with an AAV vector, an AAV capsid or a gene therapy composition. The term “contacting a cell” (e.g., contacting a cell with an AAV vector, an AAV capsid, or a gene therapy composition) as used herein, includes contacting a cell, e.g., a neuron directly or indirectly. In some aspects, contacting a cell with an AAV vector, an AAV capsid, or the gene therapy composition includes contacting a cell in vitro with the gene therapy composition, the AAV vector, or the AAV capsid or contacting a cell in vivo with the AAV vector, the AAV capsid, or the gene therapy composition. Thus, for example, the AAV vector, the AAV capsid, or the gene therapy composition can be put into physical contact with the cell by the individual performing the method, or alternatively, the AAV vector, the AAV capsid, or the gene therapy composition can be put into a situation that will permit or cause it to subsequently come into contact with the cell.

In some aspects, contacting a cell in vitro can be done, for example, by incubating the cell with the AAV vector. In some aspects, contacting a cell in vivo can be done, for example, by injecting the AAV vector, the AAV capsid, or the gene therapy composition of the disclosure into or near the tissue where the cell is located (e.g., into a trigeminal ganglion), or by injecting the AAV vector, the AAV capsid, or the gene therapy composition into another area, e.g., the bloodstream or the subcutaneous space, such that the agent will subsequently reach the tissue where the cell to be contacted is located. For example, the AAV vector can be encapsulated and/or coupled to a ligand that directs the AAV vector to a site of interest. Combinations of in vitro and in vivo methods of contacting are also possible. For example, a cell can be contacted in vitro with an AAV vector, an AAV capsid, or the gene therapy composition and subsequently transplanted into a subject.

In some aspects, contacting a cell with a polynucleotide, expression cassette, vector, rAAV particle, or composition of the disclosure includes “introducing” or “delivering” (directly or indirectly) the AAV vector, the AAV capsid, or the gene therapy composition into the cell by facilitating or effecting uptake or absorption into the cell. Introducing an AAV vector, an AAV capsid, or a gene therapy composition into a cell can be in vitro and/or in vivo. For example, for in vivo introduction, an AAV vector, an AAV capsid, a gene therapy composition can be injected into a specific tissue site (e.g., a trigeminal ganglion where a therapeutic effect is desired) or administered systemically (e.g., administering a AAV vector targeted to a locus where a therapeutic effect is desired). In vitro introduction into a cell includes methods known in the art such as electroporation and lipofection.

Provided herein are hybrid AAV5 serotype vectors. In some aspects, the hybrid AAV5 serotype vectors comprise a genome containing AAV2 inverted terminal repeats (ITRs) packaged into AAV5 serotype capsids to deliver a modified a7-GlyR LGIC (AAV5-m-α7-GlyR LGIC) to neurons of the trigeminal ganglion. In some aspects, the AAV5 capsid comprises SEQ ID NO: 9.

In some aspects, an AAV5-m-α7-GlyR LGIC is administered through intravenous, intramuscular, intracoronary, intracranial, intrathecal, intravitreal, subretinal, percutaneous, or intraarticular administration. In some aspects, the AAV5-m-α7-GlyR LGIC is SEQ ID NO: 7.

In some aspects, an AAV5-m-α7-GlyR LGIC is administered directly into the trigeminal ganglia by a focal injection.

In some aspects, an AAV5-m-α7-GlyR LGIC is administered directly into the trigeminal ganglia by a focal injection to a subject suffering from a neuralgia associated with the trigeminal nerve, more specifically a neuralgia caused by hyperexcitability of the trigeminal nerve, including trigeminal neuralgia; a trigeminal autonomic cephalgia (“TAC”) including short-lasting unilateral neuralgiform headache attack with conjunctival injection and tearing (“SUNCT”), short-lasting unilateral neuralgiform headache attack with cranial autonomic symptoms (“SUNA”), episodic cluster headache, and chronic cluster headache; trigeminal deafferation pain; burning mouth syndrome; or post-traumatic trigeminal neuropathic pain.

In some aspects, after entry into the ganglionic cell bodies, the AAV5 capsids release the encapsidated m-α7-GlyR LGIC recombinant viral genomes, which are then expressed selectively in those neurons. In some aspects, the AAV5-m-α7-GlyR LGIC is SEQ ID NO: 7. In some aspects, when activated by varenicline, the m-α7-GlyR inhibit neuronal activity, thus decreasing the hyperexcitability of trigeminal neurons and suppressing trigeminal neuralgia, trigeminal autonomic cephalgia (“TAC”) including short-lasting unilateral neuralgiform headache attack with conjunctival injection and tearing (“SUNCT”), short-lasting unilateral neuralgiform headache attack with cranial autonomic symptoms (“SUNA”), episodic cluster headache, and chronic cluster headache; trigeminal deafferation pain; burning mouth syndrome; or post-traumatic trigeminal neuropathic pain.

In the absence of varenicline, the m-α7-GlyR LGICs have no constitutive activity.

In some aspects, the AAV5 vector delivered m-α7-GlyR LGIC recombinant viral genomes can remain present in the transduced trigeminal ganglion neurons and can support expression of m-α7-GlyR LGICs in the transduced cells. In some aspects, the AAV5 vector delivered m-α7-GlyR LGIC recombinant viral genomes remain present in the transduced trigeminal ganglion neurons for at least one, two, three, four, five, or up to six months. In some aspects, expressed m-α7-GlyR LGICs can be activated as needed by administering varenicline to the subject.

In some aspects, expressed m-α7-GlyR LGICs that are activated by varenicline decrease the hyperexcitability of neurons in the trigeminal ganglia and thereby decrease pain attacks experienced by subjects with trigeminal neuralgia.

### V. Varenicline

In some aspects, varenicline is administered to subjects after administration of AAV5-m-α7-GlyR LGIC. In some aspects, varenicline is administered concomitantly as AAV5-m-α7-GlyR LGIC. In some aspects, varenicline is administered to subjects before administration of AAV5-m-α7-GlyR LGIC.

In some aspects, chronic dosing with 0.5 mg/day to 1 mg/day varenicline achieves varenicline plasma concentrations that are sufficient to activate a m-α7-GlyR LGIC (EC₅₀=1.6 nM) expressed in trigeminal ganglion neurons following intra-ganglion injection of AAV5-m-α7-GlyR LGIC. In some aspects, the AAV5-m-α7-GlyR LGIC is SEQ ID NO: 7.

In some aspects, varenicline is administered at about 0.5 mg/day, about 0.6 mg/day, about 0.7 mg/day, about 0.8 mg/day, about 0.9 mg/day, or about 1 mg/day.

In some aspects, the varenicline plasma concentration is between about 0.1 nM and about 10 nM, or about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.1 nM, about 1.2 nM, about 1.3 nM, about 1.4 nM, about 1.5 nM, about 1.6 nM, about 1.7 nM, about 1.8 nM, about 1.9 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, or about 10 nM.

In some aspects, varenicline is administered once per day. In some aspects, varenicline is administered twice per day. In some aspects, varenicline is administered three times per day.

In some aspects, when subjects being administered varenicline experience side effects, e.g., nausea, varenicline is administered at a dose of 0.5 mg once a day or varenicline administration is paused for about 1 to about 3 days and administered thereafter at 0.5 mg/day.

### V. Methods of Treatment

Provided are methods of treating trigeminal neuralgia. In some aspects, a method of treating trigeminal neuralgia comprises administering an AAV5 vector comprising a α7^{L131G, Q139L, Y217F}-GlyR LGIC transgene (AAV5-m-α7-GlyR LGIC) to a subject experiencing trigeminal neuralgia. In some aspects, the AAV5-m-α7-GlyR LGIC is SEQ ID NO: 7. In some aspects, a method of treating trigeminal neuralgia comprises administering an AAV5-m-α7-GlyR LGIC to a subject with a history of trigeminal neuralgia.

In some aspects, provided are methods of treating neuralgias associated with the trigeminal nerve, more specifically neuralgias caused by hyperexcitability of the trigeminal nerve, including trigeminal neuralgia; trigeminal autonomic cephalgias (“TACs”) including short-lasting unilateral neuralgiform headache attacks with conjunctival injection and tearing (“SUNCT”), short-lasting unilateral neuralgiform headache attacks with cranial autonomic symptoms (“SUNA”), episodic cluster headache, and chronic cluster headache; trigeminal deafferation pain; burning mouth syndrome; and post-traumatic trigeminal neuropathic pain by administering an AAV5 vector encoding a modified LGIC that selectively binds varenicline to a subject having trigeminal neuralgia and further administering varenicline to the subject. In some aspects, the AAV5 vector encoding a modified LGIC that selectively binds varenicline comprises SEQ ID NO: 7.

In some aspects, provided are methods of modulating an excitability of a neuronal cell in a subject by administering an AAV5 vector encoding a modified LGIC that selectively binds varenicline to a subject having a condition or disorder characterized by neuronal cell excitability. In some aspects, the condition or disorder is characterized by excitability of a motor neuron. In some aspects, the condition or disorder is characterized by excitability of a motor neuron. In some aspects, a condition or disorder characterized by neuronal cell excitability includes trigeminal neuralgia, a trigeminal autonomic cephalgia (“TAC”) including short-lasting unilateral neuralgiform headache attack with conjunctival injection and tearing (“SUNCT”), short-lasting unilateral neuralgiform headache attack with cranial autonomic symptoms (“SUNA”), episodic cluster headache, and chronic cluster headache; trigeminal deafferation pain; burning mouth syndrome; and post-traumatic trigeminal neuropathic pain. In some aspects, the AAV5 vector encoding a modified LGIC that selectively binds varenicline comprises SEQ ID NO: 7.

In some aspects, provided are methods of modulating an increased activity of a neuronal cell in a subject, wherein activity of a neuronal cell includes, but is not limited to, active transport (e.g., ion transport), passive transport, excitation, ion flux (e.g., calcium ion flux), and exocytosis by administering an AAV5 vector encoding a modified LGIC that selectively binds varenicline to a subject having a condition or disorder characterized by an increased activity of a neuronal cell. In some aspects, the condition or disorder is characterized by excitability of a motor neuron. In some aspects, the condition or disorder is characterized by excitability of a motor neuron. In some aspects, a condition or disorder characterized by an increased activity of a neuronal cell includes a trigeminal autonomic cephalgia (“TAC”) including short-lasting unilateral neuralgiform headache attack with conjunctival injection and tearing (“SUNCT”), short-lasting unilateral neuralgiform headache attack with cranial autonomic symptoms (“SUNA”), episodic cluster headache, and chronic cluster headache; trigeminal deafferation pain; burning mouth syndrome; and post-traumatic trigeminal neuropathic pain. In some aspects, the AAV5 vector encoding a modified LGIC that selectively binds varenicline comprises SEQ ID NO: 7.

In some aspects, provided are methods of modulating ion transport across a neural cell membrane of a subject by administering an AAV5 vector encoding a modified LGIC that selectively binds varenicline to a subject having a condition or disorder characterized by ion transport across a neural cell membrane. In some aspects, the condition or disorder is characterized by excitability of a motor neuron. In some aspects, the condition or disorder is characterized by excitability of a motor neuron. In some aspects, a condition or disorder characterized by ion transport across a neural cell membrane includes a trigeminal autonomic cephalgia (“TAC”) including short-lasting unilateral neuralgiform headache attack with conjunctival injection and tearing (“SUNCT”), short-lasting unilateral neuralgiform headache attack with cranial autonomic symptoms (“SUNA”), episodic cluster headache, and chronic cluster headache; trigeminal deafferation pain; burning mouth syndrome; and post-traumatic trigeminal neuropathic pain. In some aspects, the AAV5 vector encoding a modified LGIC that selectively binds varenicline comprises SEQ ID NO: 7.

In some aspects, a method of treating trigeminal neuralgia comprises administering an AAV5-m-α7-GlyR LGIC to a subject experiencing acute trigeminal neuralgia. In some aspects, a method of treating trigeminal neuralgia comprises administering an AAV5-m-α7-GlyR LGIC to a subject with chronic trigeminal neuralgia. In some aspects, the AAV5-m-α7-GlyR LGIC is SEQ ID NO: 7.

In some aspects, a method of treating trigeminal neuralgia comprises administering an AAV5-m-α7-GlyR LGIC to a subject with classical or idiopathic trigeminal neuralgia that has failed at least one standard of care anti-epileptic medication. In some aspects, the subject with classical or idiopathic trigeminal neuralgia that has failed at least one standard of care anti-epileptic medication with, e.g., carbamazepine, oxcarbazepine, pregabalin, gabapentin, phenytoin, or lamotrigine. In some aspects, the AAV5-m-α7-GlyR LGIC is SEQ ID NO: 7.

In some aspects, the method further comprises administering varenicline to a subject that received treatment with AAV5-m-α7-GlyR LGIC.

In some aspects, an AAV5-m-α7-GlyR LGIC is administered to a subject having trigeminal neuralgia prior to administering varenicline. In some aspects, the AAV5-m-α7-GlyR LGIC is SEQ ID NO: 7.

In some aspects, an AAV5-m-α7-GlyR LGIC is administered to a subject having trigeminal neuralgia after administration of varenicline. In some aspects, the AAV5-m-α7-GlyR LGIC is SEQ ID NO: 7.

In some aspects, an AAV5-m-α7-GlyR LGIC is administered to a subject having trigeminal neuralgia together with varenicline. In some aspects, an AAV5-m-α7-GlyR LGIC is administered to a subject having trigeminal neuralgia and varenicline is administered between about 7 days and about 4 weeks after the administration of the AAV5-m-α7-GlyR LGIC. In some aspects, the AAV5-m-α7-GlyR LGIC is SEQ ID NO: 7.

In some aspects, an AAV5-m-α7-GlyR LGIC is administered to a subject having trigeminal neuralgia, and varenicline is administered about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 30 days, or about 31 days after the administration of the AAV5-m-α7-GlyR LGIC. In some aspects, the AAV5-m-α7-GlyR LGIC is SEQ ID NO: 7

In some aspects, an AAV5-m-α7-GlyR LGIC is administered to a subject having trigeminal neuralgia, and varenicline is administered about 1 week after the administration of the AAV5-m-α7-GlyR LGIC. In some aspects, the AAV5-m-α7-GlyR LGIC is SEQ ID NO: 7.

In some aspects, an AAV5-m-α7-GlyR LGIC is administered to a subject having trigeminal neuralgia, and varenicline is administered about 2 weeks after the administration of the AAV5-m-α7-GlyR LGIC. In some aspects, the AAV5-m-α7-GlyR LGIC is SEQ ID NO: 7.

In some aspects, an AAV5-m-α7-GlyR LGIC is administered to a subject having trigeminal neuralgia, and varenicline is administered about 3 weeks after the administration of the AAV5-m-α7-GlyR LGIC. In some aspects, the AAV5-m-α7-GlyR LGIC is SEQ ID NO: 7.

In some aspects, an AAV5-m-α7-GlyR LGIC is administered once to a subject having trigeminal neuralgia, and varenicline is administered more than once.

In some aspects, an AAV5-m-α7-GlyR LGIC is administered once to a subject having trigeminal neuralgia, and varenicline is administered once a day. In some aspects, the AAV5-m-α7-GlyR LGIC is SEQ ID NO: 7.

In some aspects, an AAV5-m-α7-GlyR LGIC is administered once to a subject having trigeminal neuralgia, and varenicline is administered twice a day. In some aspects, the AAV5-m-α7-GlyR LGIC is SEQ ID NO: 7.

In some aspects, an AAV5-m-α7-GlyR LGIC is administered once to a subject having trigeminal neuralgia, and varenicline is administered three times a day. In some aspects, the AAV5-m-α7-GlyR LGIC is SEQ ID NO: 7.

In some aspects, an AAV5-m-α7-GlyR LGIC is administered once to a subject having trigeminal neuralgia, and varenicline is administered more than once beginning between about 7 days and about 4 weeks after the administration of the AAV5-m-α7-GlyR LGIC. In some aspects, the AAV5-m-α7-GlyR LGIC is SEQ ID NO: 7.

In some aspects, an AAV5-m-α7-GlyR LGIC is administered once to a subject having trigeminal neuralgia and varenicline is administered more than once beginning about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 30 days, or about 31 days after the administration of the AAV5-m-α7-GlyR LGIC. In some aspects, the AAV5-m-α7-GlyR LGIC is SEQ ID NO: 7.

In some aspects, an AAV5-m-α7-GlyR LGIC is administered once to a subject having trigeminal neuralgia and varenicline is administered more than once beginning about 1 week to about 2 weeks after the administration of the AAV5-m-α7-GlyR LGIC. In some aspects, the AAV5-m-α7-GlyR LGIC is SEQ ID NO: 7.

In some aspects, an AAV5-m-α7-GlyR LGIC is administered once to a subject having trigeminal neuralgia and varenicline is administered to the subject repeatedly for a time period of between 1 day and about 10 years or more. In some aspects, the AAV5-m-α7-GlyR LGIC is SEQ ID NO: 7.

In some aspects, varenicline is administered for about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 days or more.

In some aspects, varenicline is administered for about 50, about 55, about 60, about 65, about 70, about 75, about 80, about 85, about 90, about 95, or about 100, about 105, about 110, about 110, about 115 or about 120 days or more.

In some aspects, varenicline is administered for about 5 months, about 6 months, about 7 months, about 8 months, about 9 months, about 10 months, about 11 months, about 12 months, or more.

In some aspects, varenicline is administered for about 1.5 years, about 2 years, about 2.5 years, about 3 years, about 3.5 years, about 4 years, about 4.5 years, about 5 years, about 5.5 years, about 6 years, about 6.5 years, about 7 years, about 7.5 years, about 8 years, about 8.5 years, about 9 years, about 9.5 years or about 10 years or more.

In some aspects, varenicline is administered once a day for about 3 months followed by 1 month of treatment withdrawal and administration of varenicline after the 1 month withdrawal for a time period of between 1 day and about 10 years.

In some aspects, an AAV5-m-α7-GlyR LGIC is administered once to a subject having trigeminal neuralgia and varenicline is administered when the subject experiences trigeminal neuralgia. In some aspects, the AAV5-m-α7-GlyR LGIC is SEQ ID NO: 7.

In some aspects, an AAV5-m-α7-GlyR LGIC is administered once to a subject having trigeminal neuralgia and varenicline is administered at least once until the subject does not experience trigeminal neuralgia. In some aspects, the AAV5-m-α7-GlyR LGIC is SEQ ID NO: 7.

In some aspects, an AAV5-m-α7-GlyR LGIC is administered once to a subject having trigeminal neuralgia and varenicline is administered about 7 days after the administration of the AAV5-m-α7-GlyR LGIC once a day for the above described time periods or when the subject experiences trigeminal neuralgia. In some aspects, the AAV5-m-α7-GlyR LGIC is SEQ ID NO: 7.

In some aspects, an AAV5-m-α7-GlyR LGIC is administered by injection into a trigeminal ganglion of a subject having trigeminal neuralgia and varenicline is administered orally to the subject. In some aspects, the AAV5-m-α7-GlyR LGIC is SEQ ID NO: 7.

In some aspects, an AAV5-m-α7-GlyR LGIC is administered by injection into a trigeminal ganglion of a subject having trigeminal neuralgia and varenicline is administered by parenteral administration to the subject. In some aspects, the AAV5-m-α7-GlyR LGIC is SEQ ID NO: 7.

In some aspects, the parenteral administration of varenicline includes subcutaneous, intravenous, intramuscular, intra-arterial, intraperitoneal, intralymphatic, and injection into a tissue of an organ. In some aspects, varenicline is injected into a trigeminal ganglion.

In some aspects, an AAV5-m-α7-GlyR LGIC is administered by injection into both trigeminal ganglia of a subject having trigeminal neuralgia and varenicline is administered orally to the subject. In some aspects, the AAV5-m-α7-GlyR LGIC is SEQ ID NO: 7.

In some aspects, an AAV5-m-α7-GlyR LGIC is administered by injection into both trigeminal ganglia of a subject having trigeminal neuralgia and varenicline is administered by parenteral administration to the subject. In some aspects, the AAV5-m-α7-GlyR LGIC is SEQ ID NO: 7.

In some aspects, a method of treating trigeminal neuralgia comprises administering from about 2×10⁷ AAV5-m-α7-GlyR LGIC viral particles to about 2×10¹² AAV5-m-α7-GlyR LGIC viral particles to a subject having trigeminal neuralgia. In some aspects, the AAV5-m-α7-GlyR LGIC is SEQ ID NO: 7.

In some aspects, a method of treating trigeminal neuralgia comprises administering from about 2×10⁷, 6×10⁷, 2×10⁸, 6×10⁸, 2×10⁹, 6×10⁹, 2×10¹⁰, 6×10¹⁰, 2×10¹¹, 6×10¹¹, 2×10¹² AAV5-m-α7-GlyR LGIC viral particles to a subject having trigeminal neuralgia. In some aspects, the AAV5-m-α7-GlyR LGIC is SEQ ID NO: 7.

In some aspects, a method of treating trigeminal neuralgia comprises administering from about 1.85×10⁹ AAV5-m-α7-GlyR LGIC viral particles to about 1.85×10¹¹ AAV5-m-α7-GlyR LGIC viral particles to a subject having trigeminal neuralgia. In some aspects, the AAV5-m-α7-GlyR LGIC is SEQ ID NO: 7.

In some aspects, a method of treating trigeminal neuralgia comprises administering from about 2×10⁷ AAV5-m-α7-GlyR LGIC vector genomes (vg) to about 2×10¹² AAV5-m-α7-GlyR LGIC vg to a subject having trigeminal neuralgia. In some aspects, the AAV5-m-α7-GlyR LGIC is SEQ ID NO: 7.

In some aspects, a method of treating trigeminal neuralgia comprises administering from about 2×10⁷, 6×10⁷, 2×10⁸, 6×10⁸, 2×10⁹, 6×10⁹, 2×10¹⁰, 6×10¹⁰, 2×10¹¹, 6×10¹¹, 2×10¹² AAV5-m-α7-GlyR LGIC vg to a subject having trigeminal neuralgia. In some aspects, the AAV5-m-α7-GlyR LGIC is SEQ ID NO: 7.

In some aspects, a method of treating trigeminal neuralgia comprises administering from about 1.85×10⁹ AAV5-m-α7-GlyR LGIC vector genomes to about 1.85×10¹¹ AAV5-m-α7-GlyR LGIC vg to a subject having trigeminal neuralgia. In some aspects, the AAV5-m-α7-GlyR LGIC is SEQ ID NO: 7.

In some aspects, a method of treating trigeminal neuralgia comprises administering an amount of an AAV5-m-α7-GlyR LGIC vector to a trigeminal ganglion of a subject having trigeminal neuralgia, which amount is sufficient to lead to an AAV5-m-α7-GlyR LGIC vector genome amount from about 1.2×10⁷ vg per mm3 trigeminal ganglion tissue to about 1.3×10⁹ vg per mm3 trigeminal ganglion tissue. In some aspects, the AAV5-m-α7-GlyR LGIC is SEQ ID NO: 7.

In some aspects, a method of treating trigeminal neuralgia comprises administering an amount of an AAV5-m-α7-GlyR LGIC vector to a trigeminal ganglion of a subject having trigeminal neuralgia, which amount is sufficient to lead to an AAV5-m-α7-GlyR LGIC vector genome amount from about 1.2×10⁷ vg per mm³, 1.3×10⁷ vg per mm³, 1.4×10⁷ vg per mm³, 1.5×10⁷ vg per mm³, 1.6×10⁷ vg per mm³, 1.7×10⁷ vg per mm³, 1.8×10⁷ vg per mm³, 1.9×10⁷ vg per mm³, about 2×10⁷ vg per mm³, 2.1×10⁷ vg per mm³, 2.2×10⁷ vg per mm³, 2.3×10⁷ vg per mm³, 2.4×10⁷ vg per mm³, 2.5×10⁷ vg per mm³, 2.6×10⁷ vg per mm³, 2.7×10⁷ vg per mm³, 2.8×10⁷ vg per mm³, 2.9×10⁷ vg per mm³, about 3×10⁷ vg per mm³, 3.1×10⁷ vg per mm³, 3.2×10⁷ vg per mm³, 3.3×10⁷ vg per mm³, 3.4×10⁷ vg per mm³, 3.5×10⁷ vg per mm³, 3.6×10⁷ vg per mm³, 3.7×10⁷ vg per mm³, 3.8×10⁷ vg per mm³, 3.9×10⁷ vg per mm³, about 4×10⁷ vg per mm³, 4.1×10⁷ vg per mm³, 4.2×10⁷ vg per mm³, 4.3×10⁷ vg per mm³, 4.4×10⁷ vg per mm³, 4.5×10⁷ vg per mm³, 4.6×10⁷ vg per mm³, 4.7×10⁷ vg per mm³, 4.8×10⁷ vg per mm³, 4.9×10⁷ vg per mm³, about 5×10⁷ vg per mm³, 5.1×10⁷ vg per mm³, 5.2×10⁷ vg per mm³, 5.3×10⁷ vg per mm³, 5.4×10⁷ vg per mm³, 5.5×10⁷ vg per mm³, 5.6×10⁷ vg per mm³, 5.7×10⁷ vg per mm³, 5.8×10⁷ vg per mm³, 5.9×10⁷ vg per mm³, about 6×10⁷ vg per mm³, 6.1×10⁷ vg per mm³, 6.2×10⁷ vg per mm³, 6.3×10⁷ vg per mm³, 6.4×10⁷ vg per mm³, 5.5×10⁷ vg per mm³, 6.6×10⁷ vg per mm³, 6.7×10⁷ vg per mm³, 6.8×10⁷ vg per mm³, 6.9×10⁷ vg per mm³, about 7×10⁷ vg per mm³, 7.1×10⁷ vg per mm³, 7.2×10⁷ vg per mm³, 7.3×10⁷ vg per mm³, 7.4×10⁷ vg per mm³, 7.5×10⁷ vg per mm³, 7.6×10⁷ vg per mm³, 7.7×10⁷ vg per mm³, 7.8×10⁷ vg per mm³, 7.9×10⁷ vg per mm³, about 8×10⁷ vg per mm³, 8.1×10⁷ vg per mm³, 8.2×10⁷ vg per mm³, 8.3×10⁷ vg per mm³, 8.4×10⁷ vg per mm³, 8.5×10⁷ vg per mm³, 8.6×10⁷ vg per mm³, 8.7×10⁷ vg per mm³, 8.8×10⁷ vg per mm³, 8.9×10⁷ vg per mm³, about 9×10⁷ vg per mm³, 9.1×10⁷ vg per mm³, 9.2×10⁷ vg per mm³, 9.3×10⁷ vg per mm³, 9.4×10⁷ vg per mm³, 9.5×10⁷ vg per mm³, 9.6×10⁷ vg per mm³, 9.7×10⁷ vg per mm³, 9.8×10⁷ vg per mm³, 9.9×10⁷ vg per mm³, about 1×10⁸ vg per mm³, about 1.1×108 vg per mm³, about 1.2×10⁸ vg per mm³, or about 1.3×10⁸ vg per mm³, about 1×10⁹ vg per mm³, about 1.1×10⁹ vg per mm³, about 1.2×10⁹ vg per mm³, or about 1.3×10⁹ vg per mm³. In some aspects, the AAV5-m-α7-GlyR LGIC is SEQ ID NO: 7.

In some aspects, a method of treating trigeminal neuralgia comprises administering an AAV5-m-α7-GlyR LGIC and varenicline. In some aspects, the AAV5-m-α7-GlyR LGIC is SEQ ID NO: 7. In some aspects, varenicline is administered orally to the subject at a dose of 0.5 mg or 1 mg once a day. In some aspects, varenicline is administered orally at a dose of 0.5 mg or 1 mg twice a day. In some aspects, varenicline is administered orally at a dose of 1 mg once a day for about 5 days or more and varenicline is administered at a dose of 0.5 mg once a day thereafter. In some aspects, varenicline is administered orally at a dose of 1 mg once a day for about 3 days or more and varenicline is administered at a dose of 0.5 mg once a day thereafter.

In some aspects, varenicline is administered orally at a dose of 0.5 mg once a day for about 5 days or more and varenicline is administered at a dose of 1 mg once a day thereafter. In some aspects, varenicline is administered orally at a dose of 0.5 mg once a day for about 5 days and varenicline is administered at a dose of 1 mg once a day thereafter. In some aspects, varenicline is administered orally at a dose of 0.5 mg once a day for about 3 days or more and varenicline is administered at a dose of 1 mg once a day thereafter. In some aspects, varenicline is administered orally at a dose of 0.5 mg once a day for about 3 days and varenicline is administered at a dose of 1 mg once a day thereafter.

In some aspects, varenicline is administered orally at a dose of 1 mg once a day until the subject does not experience trigeminal neuralgia and varenicline is administered thereafter at a dose of 0.5 mg once a day. In some aspects, when a subject experiences trigeminal neuralgia while being treated orally with 0.5 mg varenicline, the administration of varenicline is increased to orally 1 mg per day and is administered until the subject does not experience trigeminal neuralgia.

In some aspects, provided is a method for controlling trigeminal pain attacks in a subject suffering from trigeminal neuralgia, the method comprising administered an AAV5-m-α7-GlyR LGIC by injection into one or both trigeminal ganglia of the subject and administering varenicline orally to the subject at a dose of 1 mg once a day until the subject does not experience trigeminal pain attacks. In some aspects, the method further comprises administering varenicline at a dose of 0.5 mg once a day for maintenance once the trigeminal pain attacks have been controlled. In some aspects, a method for controlling trigeminal pain attacks comprises administering an AAV5-m-α7-GlyR LGIC to a subject having trigeminal neuralgia pain attacks and administering varenicline between about 7 days and about 4 weeks after the administration of the AAV5-m-α7-GlyR LGIC. In some aspects, the AAV5-m-α7-GlyR LGIC is SEQ ID NO: 7.

In some aspects, an AAV5-m-α7-GlyR LGIC is administered to a subject having trigeminal neuralgia pain attacks and varenicline is administered about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 30 days, or about 31 days after the administration of the AAV5-m-α7-GlyR LGIC. In some aspects, the AAV5-m-α7-GlyR LGIC is SEQ ID NO: 7.

In some aspects, a method of controlling trigeminal neuralgia pain attacks comprises administering an AAV5-m-α7-GlyR LGIC once to a subject having trigeminal neuralgia pain attacks and administering varenicline to the subject repeatedly for a time period of between 1 day and 10 years or more. In some aspects, varenicline is administered for about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 days or more. In some aspects, a method of controlling trigeminal neuralgia pain attacks comprises administering an AAV5-m-α7-GlyR LGIC once to a subject having trigeminal neuralgia pain attacks and administering varenicline to the subject until the pain attacks subside. In some aspects, the AAV5-m-α7-GlyR LGIC is SEQ ID NO: 7.

In some aspects, a method of treating trigeminal neuralgia pain attacks comprises administering from about 2×10⁷, 6×10⁷, 2×10⁸, 6×10⁸, 2×10⁹, 6×10⁹, 2×10¹⁰, 6×10¹⁰, 2×10¹¹, 6×10¹¹, 2×10¹² AAV5-m-α7-GlyR LGIC viral particles to a subject having trigeminal neuralgia pain attacks. In some aspects, the AAV5-m-α7-GlyR LGIC is SEQ ID NO: 7.

In some aspects, a method of treating trigeminal neuralgia pain attacks comprises administering an amount of an AAV5-m-α7-GlyR LGIC vector to a trigeminal ganglion of a subject having trigeminal neuralgia pain attacks, which amount is sufficient to lead to an AAV5-m-α7-GlyR LGIC vector genome amount from about 1.2×10⁷ vg per mm³ trigeminal ganglion tissue to about 1.3×10⁹ vg per mm³ trigeminal ganglion tissue. In some aspects, the AAV5-m-α7-GlyR LGIC is SEQ ID NO: 7.

In some aspects, a method of treating trigeminal neuralgia pain attacks comprises administering an AAV5-m-α7-GlyR LGIC and varenicline. In some aspects, varenicline is administered orally to the subject experiencing pain attacks at a dose of 0.5 mg or 1 mg once a day. In some aspects, varenicline is administered orally at a dose of 0.5 mg or 1 mg twice a day. In some aspects, varenicline is administered orally at a dose of 1 mg once a day for about 1 day or until the pain attacks subside and varenicline is administered at a dose of 0.5 mg once a day thereafter. In some aspects, varenicline is administered orally at a dose of 1 mg once a day for about 3 days or more until the pain attacks subside and varenicline is administered at a dose of 0.5 mg once a day thereafter. In some aspects, varenicline is administered orally at a dose of 1 mg once a day for about 3 days or more and varenicline is administered at a dose of 0.5 mg once a day thereafter. In some aspects, varenicline is administered orally at a dose of 1 mg once a day for as many days as the pain attacks occur and varenicline is administered at a dose of 0.5 mg once a day thereafter. In some aspects, varenicline is administered orally at a dose of 1 mg once a day for as many days as the pain attacks occur and varenicline is administered thereafter only upon reoccurrence of the pain attacks. In some aspects, the AAV5-m-α7-GlyR LGIC is SEQ ID NO: 7.

In some aspects, provided is a method for controlling acute trigeminal pain attacks in a subject suffering from trigeminal neuralgia, the method comprising administered an AAV5-m-α7-GlyR LGIC by injection into one or both trigeminal ganglia of the subject and administering varenicline parenteral to the subject at a dose of 1 mg once a day until the subject does not experience trigeminal pain attacks. In some aspects, the AAV5-m-α7-GlyR LGIC is SEQ ID NO: 7.

In some aspects, the method further comprises administering varenicline at a dose of 0.5 mg once a day either parenteral or oral for maintenance once the trigeminal pain attacks have been controlled. In some aspects, the AAV5-m-α7-GlyR LGIC is SEQ ID NO: 7.

In some aspects, provided is a method for controlling chronic trigeminal neuralgia in a subject suffering from chronic trigeminal neuralgia, the method comprising administered an AAV5-m-α7-GlyR LGIC by injection into one or both trigeminal ganglia of the subject and administering varenicline orally to the subject at a dose of 0.5 mg or 1 mg once a day to control the chronic trigeminal neuralgia. In some aspects, the AAV5-m-α7-GlyR LGIC is SEQ ID NO: 7.

In some aspects, provided is a method for controlling chronic trigeminal neuralgia in a subject suffering from chronic trigeminal neuralgia, the method comprising administered an AAV5-m-α7-GlyR LGIC by injection into one or both trigeminal ganglia of the subject and administering varenicline parenteral to the subject at a dose of 0.5 mg or 1 mg once a day to control the chronic trigeminal neuralgia. In some aspects, the AAV5-m-α7-GlyR LGIC is SEQ ID NO: 7.

In some aspects, provided is a method for treating trigeminal neuralgia in a subject suffering from trigeminal neuralgia, the method comprising administering an AAV5-m-α7-GlyR LGIC by injection into one or both trigeminal ganglia of the subject and administering varenicline at any time when the subject experiences trigeminal neuralgia. In some aspects, the AAV5-m-α7-GlyR LGIC is SEQ ID NO: 7.

In some aspects, varenicline is administered at a dose of 1 mg once a day until the subject does not experience trigeminal neuralgia. In some aspects, varenicline is administered at a dose of 0.5 mg once a day until the subject does not experience trigeminal neuralgia. In some aspects, varenicline is administered at a dose of 0.5 mg or 1 mg once a day when the subject experiences trigeminal neuralgia again. In some aspects, varenicline is administered at a dose of 0.5 mg or 1 mg twice a day when the subject experiences trigeminal neuralgia again. In some aspects, the methods described herein further comprise treating the subject with varenicline until the subject does not experience trigeminal neuralgia. In some aspects, the AAV5-m-α7-GlyR LGIC is SEQ ID NO: 7.

In some aspects, provided is a method for treating trigeminal neuralgia in a subject suffering from trigeminal neuralgia, the method comprising administering a first dose of an AAV5-m-α7-GlyR LGIC by injection into one or both trigeminal ganglia of the subject and administering varenicline to the subject In some aspects, the AAV5-m-α7-GlyR LGIC is SEQ ID NO: 7.

In some aspects, the method further comprises administering a second dose of an AAV5-m-α7-GlyR LGIC by injection into one or both trigeminal ganglia of the subject and administering varenicline. In some aspects, the AAV5-m-α7-GlyR LGIC is SEQ ID NO: 7.

In some aspects, the method further comprises administering more than two doses of an AAV5-m-α7-GlyR LGIC by injection into one or both trigeminal ganglia of the subject and administering varenicline. In some aspects, the AAV5-m-α7-GlyR LGIC is SEQ ID NO: 7.

In some aspects, provided is a method for treating trigeminal neuralgia in a subject who experiences trigeminal neuralgia after a first injection of AAV5-m-α7-GlyR LGIC into one or both trigeminal ganglia and after administration of varenicline at a dose of up to 1 mg twice per day, the method comprising administering a second dose of AAV5-m-α7-GlyR LGIC by injection into one or both trigeminal ganglia and administering varenicline thereafter at a dose of 0.5 mg or 1 mg once per day to the subject. In some aspects, the AAV5-m-α7-GlyR LGIC is SEQ ID NO: 7.

In some aspects, the method of treating trigeminal neuralgia comprises administering an AAV5-m-α7-GlyR LGIC to a subject suffering from trigeminal neuralgia once by direct injection into a trigeminal ganglion and administering varenicline to the subject upon occurrence of trigeminal pain. In some aspects, the AAV5-m-α7-GlyR LGIC is SEQ ID NO: 7

In some aspects, the method of treating trigeminal neuralgia comprises administering an AAV5-m-α7-GlyR LGIC to a subject suffering from trigeminal neuralgia once by direct injection into the trigeminal ganglion on the side of the face on which the trigeminal pain occurs and administering varenicline to the subject upon occurrence of trigeminal pain. In some aspects, the AAV5-m-α7-GlyR LGIC is SEQ ID NO: 7.

In some aspects, the method of treating trigeminal neuralgia comprises administering an AAV5-m-α7-GlyR LGIC to a subject suffering from trigeminal neuralgia once by direct injection into each trigeminal ganglion and administering varenicline to the subject upon occurrence of trigeminal pain. In some aspects, both trigeminal ganglia are injected with AAV5-mLGIC even though the pain mainly is triggered in one trigeminal ganglion. In some aspects, the AAV5-m-α7-GlyR LGIC is SEQ ID NO: 7.

In some aspects, the method of treating trigeminal neuralgia comprises administering an AAV5-m-α7-GlyR LGIC to a subject suffering from trigeminal neuralgia once by direct injection into the trigeminal ganglion on the side of the face on which the trigeminal pain occurs and administering varenicline to the subject upon occurrence of trigeminal pain. In some aspects, the method further comprises administering a second dose of an AAV5-m-α7-GlyR LGIC by direct injection into the trigeminal ganglion on the opposite side of the face on which the trigeminal pain occurs and administering varenicline to the subject upon occurrence of trigeminal pain. In some aspects, a second injection of an AAV5-m-α7-GlyR LGIC is performed only after trigeminal pain reoccurred following a first injection of an AAV5-m-α7-GlyR LGIC. In some aspects, the AAV5-m-α7-GlyR LGIC is SEQ ID NO: 7.

In some aspects, the method of treating trigeminal neuralgia comprises administering an AAV5-m-α7-GlyR LGIC to a subject suffering from trigeminal neuralgia once by direct injection into a trigeminal ganglion and administering varenicline to the subject starting from about 7 days to about 21 days after the AAV5-m-α7-GlyR LGIC injection. In some aspects, the AAV5-m-α7-GlyR LGIC is SEQ ID NO: 7.

In some aspects, the method of treating trigeminal neuralgia comprises administering an AAV5-m-α7-GlyR LGIC to a subject suffering from trigeminal neuralgia once by direct injection into a trigeminal ganglion and administering varenicline to the subject starting from about 14 days after the AAV5-m-α7-GlyR LGIC injection. In some aspects, the AAV5-m-α7-GlyR LGIC is SEQ ID NO: 7.

In some aspects, the combination treatment with AAV5-m-α7-GlyR LGIC and varenicline can be administered more than once without eliciting adverse immune reactions, e.g., against the AAV5-m-α7-GlyR LGIC vectors. In some aspects, the direct injection of AAV5-m-α7-GlyR LGIC vector into one or both trigeminal ganglia prevents anti-AAV5 antibody formation prevents a subsequent treatment with AAV5-m-α7-GlyR LGIC vectors. In some aspects, the AAV5-m-α7-GlyR LGIC is SEQ ID NO: 7.

In some aspects, AAV5-m-α7-GlyR LGIC vector genomes can be present as episomal genomes in the injected cells. In some aspects, the methods described herein can provide a continuous expression of m-α7-GlyR LGICs such as to continuously treat trigeminal neuralgia by the administration of varenicline. In some aspects, the AAV5-m-α7-GlyR LGIC is SEQ ID NO: 7.

### Pharmaceutical Compositions and Kits

Certain aspects of the disclosure are directed to a pharmaceutical composition comprising any of the synthetic promoters disclosed herein, any of the polynucleotides disclosed herein, any of the expression constructs disclosed herein, any of the delivery vectors disclosed herein, or any of the viral particles (e.g., rAAV) disclosed herein.

In some aspects, the pharmaceutical further comprises a pharmaceutically acceptable excipient.

Pharmaceutically acceptable excipients or carriers are determined in part by the particular composition being administered, as well as by the particular method used to administer the composition.

Accordingly, there is a wide variety of suitable formulations of pharmaceutical compositions comprising a delivery vector of the present disclosure (e.g., an AAV vector) or a plurality thereof (see, e.g., Remington's Pharmaceutical Sciences, Mack Publishing Co., Easton, Pa. 18th ed. (1990)) and/or one or more shRNA disclosed herein. The pharmaceutical compositions are generally formulated sterile and in full compliance with all Good Manufacturing Practice (GMP) regulations of the U.S. Food and Drug Administration. In some aspects, the pharmaceutical composition comprises more than one AAV vector of the present disclosure, wherein each vector comprises at least one modified chimeric LGIC disclosed herein.

Provided herein are formulations comprising an AAV5-m-α7-GlyR LGIC at a concentration of about 0.8 to about 5.0×10¹³ vector genomes per milliliter (vg/mL) in a formulation buffer. In some aspects, the formulation comprises an AAV5-m-α7-GlyR LGIC vector at a concentration of about 0.8 to about 5.0×10¹² vg/mL. In some aspects, the AAV5-m-α7-GlyR LGIC is SEQ ID NO: 7.

In some aspects, a formulation comprises an AAV5-m-α7-GlyR LGIC vector at a concentration of about 8×10¹¹ vg/mL, about 8.2×10¹¹ vg/mL, about 8.4×10¹¹ vg/mL, about 8.6×10¹¹ vg/mL, about 8.8×10¹¹ vg/mL, about 9×10¹¹ vg/mL, about 9.2×10¹¹ vg/mL, about 9.4×10¹¹ vg/mL, about 9.6×10¹¹ vg/mL, about 9.8×10¹¹ vg/mL, about 1×10¹² vg/mL, about 1.2×10¹² vg/mL, about 1×10¹² vg/mL, about 1.4×10¹² vg/mL, about 1.6×10¹² vg/mL, about 1.8×10¹² vg/mL, about 2×10¹² vg/mL, about 2.2×10¹² vg/mL, about 2.4×10¹² vg/mL, about 2.6×10¹² vg/mL, about 2.8×10¹² vg/mL, about 3×10¹² vg/mL, about 3.2×10¹² vg/mL, about 3.4×10¹² vg/mL, about 3.6×10¹² vg/mL, about 3.8×10¹² vg/mL, about 4×10¹² vg/mL,, about 4.2×10¹² vg/mL, about 4.4×10¹² vg/mL, about 4.6×10¹² vg/mL, about 4.8×10¹² vg/mL, about 5×10¹² vg/mL, about 5.2×10¹² vg/mL, about 5.4×10¹² vg/mL, about 5.6×10¹² vg/mL, about 5.8×10¹² vg/mL, about 6×10¹² vg/mL, about 6.2×10¹² vg/mL, about 6.4×10¹² vg/mL, about 6.6×10¹² vg/mL, about 6.8×10¹² vg/mL, about 7×10¹² vg/mL, about 7.2×10¹² vg/mL, about 7.4×10¹² vg/mL about 7.6×10¹² vg/mL, about 7.8×10¹² vg/mL, about 8×10¹² vg/mL, about 8.2×10¹² vg/mL, about 8.4×10¹² vg/mL, about 8.6×10¹² vg/mL, about 8.8×10¹² vg/mL, about 9×10¹² vg/mL, about 9.2×10¹² vg/mL, about 9.4×10¹² vg/mL, about 9.6×10¹² vg/mL, about 9.8×10¹² vg/mL, about 1×10¹³ vg/mL, about 1.2×10¹³ vg/mL, about 1.4×10¹³ vg/mL, about 1.6×10¹³ vg/mL, about 1.8×10¹³ vg/mL, about 2×10¹³ vg/mL, about 2.2×10¹³ vg/mL, about 2.4×10¹³ vg/mL, about 2.6×10¹³ vg/mL, about 2.8×10¹³ vg/mL, about 3×10¹³ vg/mL, about 3.2×10¹³ vg/mL, about 3.4×10¹³ vg/mL, about 3.6×10¹³ vg/mL, about 3.8×10¹³ vg/mL, about 4×10¹³ vg/mL, about 4.2×10¹³ vg/mL, about 4.4×10¹³ vg/mL, about 4.6×10¹³ vg/mL, about 4.8×10¹³ vg/mL, or about 5×10¹³ vg/mL.

In some aspects, a formulation buffer comprises a sodium phosphate buffer. In some aspects, a formulation buffer further comprises sodium chloride. In some aspects, a formulation buffer further comprises a non-ionic co-polymer.

In some aspects, a formulation buffer comprises from about 1 mM to about 20 mM sodium phosphate buffer. In some aspects, a formulation buffer comprises 10 mM sodium phosphate buffer. In some aspects, a formulation buffer comprises 2 mM sodium dihydrogen phosphate (NaH₂PO₄). In some aspects, a formulation buffer comprises 8 mM disodium hydrogen phosphate (Na₂HPO₄).

In some aspects, a formulation buffer further comprises from about 120 mM to about 240 mM sodium chloride. In some aspects, a formulation buffer further comprises 180 mM sodium chloride.

In some aspects, a formulation buffer further comprises poloxamer 188. In some aspects, a formulation buffer comprises 0.0005% to about 0.005% poloxamer 188 (Pluronic™ F-68). In some aspects, a formulation buffer comprises 0.001% poloxamer 188.

In some aspects, a formulation buffer has a pH of about 6.8 to about 7.8. In some aspects, a formulation buffer has a pH 7.3±0.2.

In some aspects, a composition described herein comprises a formulation with 10 mM sodium phosphate buffer, 180 mM sodium chloride, and 0.001% poloxamer 188, and has a pH 7.3±0.2.

In some aspects, a composition described herein comprises an AAV5-m-α7-GlyR LGIC vector at a concentration of about 0.8 to about 5.0×10¹³ vg/mL in a formulation with 10 mM sodium phosphate buffer, 180 mM sodium chloride, and 0.001% poloxamer 188, and has a pH 7.3±0.2. In some aspects, the AAV5-m-α7-GlyR LGIC is SEQ ID NO: 7.

In some aspects, a composition described herein comprises a formulation buffer and an AAV5-m-α7-GlyR LGIC vector at a concentration of about 0.8×10¹² to about 5.0×10¹² vg/mL. In some aspects, the AAV5-m-α7-GlyR LGIC is SEQ ID NO: 7.

In some aspects, a composition comprises a formulation buffer and an AAV5-m-α7-GlyR LGIC vector at a concentration of about 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ AAV5-m-α7-GlyR LGIC vg. In some aspects, the AAV5-m-α7-GlyR LGIC is SEQ ID NO: 7.

In some aspects, a composition comprises an AAV5-m-α7-GlyR LGIC vector at a concentration of about 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ AAV5-m-α7-GlyR LGIC vg and a formulation buffer comprising from about 1 mM to about 20 mM sodium phosphate buffer. In some aspects, the AAV5-m-α7-GlyR LGIC is SEQ ID NO: 7.

In some aspects, a composition comprises an AAV5-m-α7-GlyR LGIC vector at a concentration of about 1×10⁷, 2×10⁷, 3×10⁷, 4×10⁷, 5×10⁷, 6×10⁷, 7×107, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ AAV5-m-α7-GlyR LGIC vg and a formulation buffer comprising from about 120 mM to about 240 mM sodium chloride. In some aspects, the AAV5-m-α7-GlyR LGIC is SEQ ID NO: 7.

In some aspects, a composition comprises an AAV5-m-α7-GlyR LGIC vector at a concentration of about 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ AAV5-m-α7-GlyR LGIC vg and a formulation buffer comprising from about 0.0005% to about 0.005% poloxamer 188. In some aspects, the AAV5-m-α7-GlyR LGIC is SEQ ID NO: 7.

In some aspects, a composition comprises an AAV5-m-α7-GlyR LGIC vector at a concentration of about 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ AAV5-m-α7-GlyR LGIC vg and a formulation buffer comprising 10 mM sodium phosphate buffer, 180 mM sodium chloride, and 0.001% poloxamer 188, and having a pH 7.3±0.2. In some aspects, the AAV5-m-α7-GlyR LGIC is SEQ ID NO: 7.

In some aspects, a composition comprises a formulation buffer and an AAV5-m-α7-GlyR LGIC vector at a concentration of about 8×10¹¹ to about 5×10¹² vg/mL. In some aspects, the AAV5-m-α7-GlyR LGIC is SEQ ID NO: 7. In some aspects, a formulation comprises about 0.8-5.0×10¹² vg/mL of an AAV5-m-α7-GlyR LGIC vector, 10 mM sodium phosphate buffer, 180 mM sodium chloride, and 0.001% poloxamer 188, and has a pH 7.3±0.2. In some aspects, the AAV5-m-α7-GlyR LGIC is SEQ ID NO: 7.

In some aspects, a composition comprises varenicline tablets for oral administration. In some aspects, a varenicline tablet comprises 0.5 mg varenicline tartrate. In some aspects, a varenicline tablet comprises 1.0 mg varenicline tartrate.

Further provided is a kit comprising a composition comprising an AAV5-m-α7-GlyR LGIC vector, a formulation buffer, varenicline tablets, and, optionally, instructions for mixing the AAV5-m-α7-GlyR LGIC vector with the formulation buffer and administering the AAV5-m-α7-GlyR LGIC vector/formulation buffer mixture to a subject by injection into a trigeminal ganglion and orally administering the varenicline tablet. In some aspects, the AAV5-m-α7-GlyR LGIC is SEQ ID NO: 7.

In some aspects, a kit comprises a composition comprising an AAV5-m-α7-GlyR LGIC vector, 10 mM sodium phosphate buffer, 180 mM sodium chloride (NaCl), and 0.001% poloxamer 188, and has a pH 7.3±0.2. In some aspects, the AAV5-m-α7-GlyR LGIC is SEQ ID NO: 7.

### EXAMPLES

### Example 1

### Formulations

The AAV5-m-α7-GlyR LGIC batch formulation is composed of AAV5-m-α7-GlyR LGIC vector at a concentration of 0.8-5.0×10¹³ vg/mL in formulation buffer. The formulation buffer consists of 10 mM sodium phosphate buffer (2 mM sodium dihydrogen phosphate [NaH₂PO₄] and 8 mM disodium hydrogen phosphate [Na₂HPO_{4]}), 180 mM sodium chloride (NaCl), and 0.001% poloxamer 188 (Pluronic™M F-68), pH 7.3±0.2. The final vector drug product is formulated at a target concentration of 0.8-5.0×10¹² vg/mL. Varenicline is supplied as 0.5 mg and 1.0 mg tablets for oral administration.

### Example 2

### Trigeminal Neuralgia Animal Model

Clinical data suggest that pain attacks in trigeminal neuralgia are triggered and amplified by increased excitability of trigeminal ganglion neurons (Devor, Clin J Pain 18 (1): 4-13, 2022), which can be a consequence of nerve injury and demyelination resulting, e.g., from a compressed trigeminal nerve. An animal model of trigeminal neuralgia was used to test the AAV5-m-α7-GlyR LGIC and varenicline combination treatment.

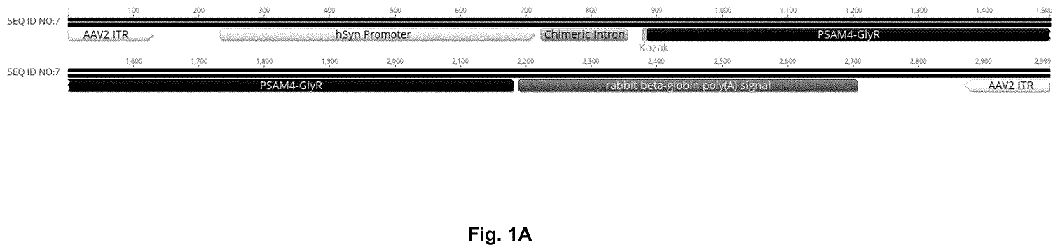

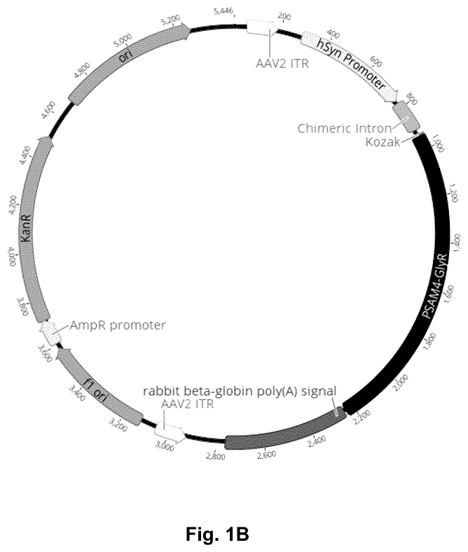

Infraorbital nerve (IoN) chronic constriction injury (IoN-CCI) is an art-recognized animal model of trigeminal neuralgia (Vos et al. J Neurosci 14 (5 Pt 1): 2708-2723, 1994). Using the rat IoN-CCI model, the efficacy and durability of a combination therapy comprising an AAV5 vector carrying a modified m-α7-GlyR LGIC transgene (AAV5-m-α7-GlyR LGIC) (FIG. 1A) in combination with varenicline (FIG. 1B) on pain sensitivity was tested.

Mechanical allodynia was measured using a von Frey filament assay to define the escape threshold [ET]) in the rat IoN-CCI model. The total study duration was 6 months with two intermittent time points (2-and 4-months). Behavioral efficacy, as assessed by ET, and transgene expression in the trigeminal ganglion were evaluated.

Twelve groups of male and female adult rats were treated. AAV5-m-α7-GlyR LGIC vectors were injected percutaneously into the left trigeminal ganglion whereas the right untreated trigeminal ganglion served as control.

Naïve animals (no IoN-CCI, no AAV5-m-α7-GlyR LGIC or varenicline dosing) served as intact controls for behavioral and molecular analysis. Two treatment paradigms were evaluated: (1) groups 2 to 4 received IoN-CCI surgery 2 weeks prior to AAV5-m-α7-GlyR LGIC injection, while (2) groups 7 to 12 were injected with AAV5-m-α7-GlyR LGIC 2 weeks prior to IoN-CCI surgery. Groups 5-6 were injected with AAV5-m-α7-GlyR LGIC diluent 2 weeks prior to IoN-CCI. Group 7 was administered vehicle in place of varenicline.

Varenicline tartrate was administered orally. A tartrate correction factor of 1.71 was applied in dose formulations.

Varenicline dosing began 4-weeks post AAV5-m-α7-GlyR LGIC injections. Animals in group 11 were administered varenicline (0.1 mg/kg) daily for 3 months followed by 1 month of treatment withdrawal; the varenicline dosing in this group was initiated again for another month until animals were sacrificed for molecular analysis. Rats in group 12 were injected with AAV5-m-α7-GlyR LGIC and allowed to express the transgene for 5 months before the IoN-CCI surgery. The varenicline dosing in this group was initiated 2 weeks post ION-CCI surgery and lasted for 2 weeks until animals were sacrificed.

Animals euthanized at 2 months were given a single daily dose of 0.1 mg/kg varenicline for 1 month (the duration of m-α7-GlyR LGIC expression was 2 months in this group). Animals euthanized at 4 months were given a single daily dose of 0.1 mg/kg varenicline for 3 months (the duration of m-α7-GlyR LGIC expression was 4 months). Animals euthanized at 6 months were given a single daily dose of 0.1 mg/kg varenicline for 5 months (the duration of m-α7-GlyR LGIC expression equaled 6 month). Efficacy presented below is a combination of all 3 time points (2, 4, and 6 months) for each treatment group.

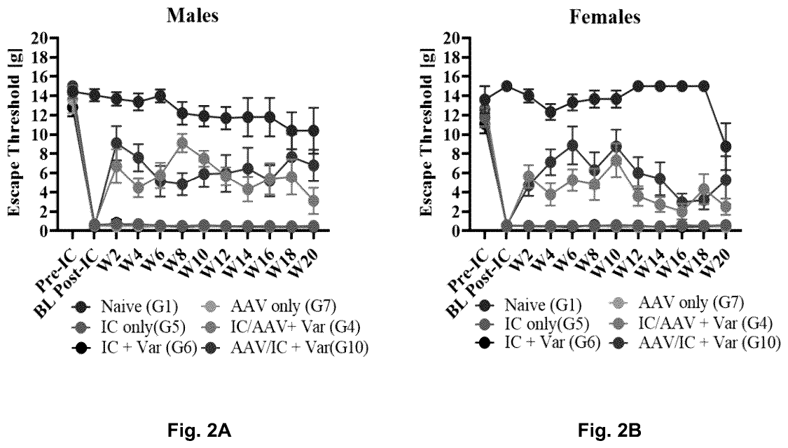

The baseline escape threshold (ET) prior to IoN-CCI surgery (Pre-IC), baseline ET after IoN-CCI surgery (BL Post-IC); and ET at weeks 2-20 post-varenicline dose are shown in FIG. 2A. Naïve (G1) animals did not receive IoN-CCI surgery, AAV5-m-α7-GlyR LGIC, or varenicline dosing. IC/AAV+Var group 4 animals received IoN-CCI surgery followed by AAV5-m-α7-GlyR LGIC (2×10⁹ vg) injection and oral varenicline treatment 1 month post AAV5-m-α7-GlyR LGIC injection. IC only group 5 animals received IoN-CCI only (without AAV5-m-α7-GlyR LGIC or varenicline). IC+Var group 6 animals received IoN-CCI surgery and varenicline (but no AAV5-m-α7-GlyR LGIC injections). AAV only group 7 animals received AAV5-m-α7-GlyR LGIC (6×10⁸ vg) followed by IoN-CCI surgery (but no varenicline treatment). AAV/IC+Var group 10 animals received AAV5-m-α7-GlyR LGIC (2×10⁹ vg) followed by IoN-CCI surgery and varenicline treatment 1 month post AAV5-m-α7-GlyR LGIC injection.

AAV5-m-α7-GlyR LGIC in combination with varenicline significantly increased ET in both male and female rats regardless of whether the animals received AAV5-m-α7-GlyR LGIC before the ION-CCI surgery or thereafter (FIGS. 2A and 2B). In contrast, neither varenicline alone nor AAV5-m-α7-GlyR LGIC alone (Groups 6 and 7, respectively) demonstrated improved pain sensitivity throughout the duration of the study (FIGS. 2A and 2B).

### EXAMPLE 3

### No Differences in Efficacy of AAV5-m-α7-GlyR LGIC Between Male and Female Rats

Next, male and female rats in the same treatment groups (IoN-CCI/AAV5-m-α7-GlyR LGIC or AAV5-m-α7-GlyR LGIC/IoN-CCI) were compared to evaluate a possible sex difference in AAV5-m-α7-GlyR LGIC-varenicline effect on ET.

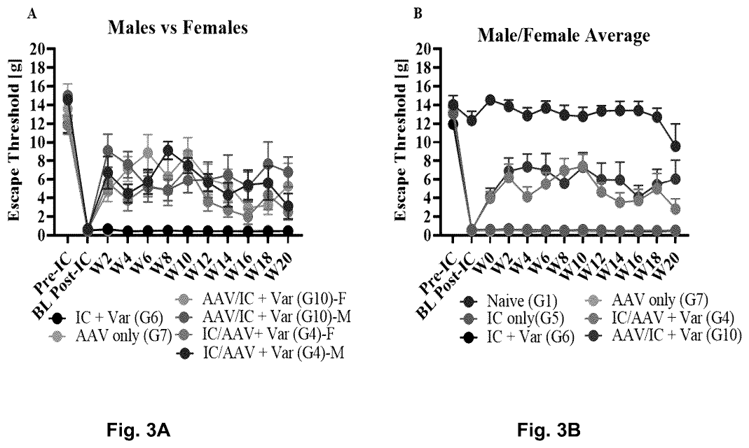

The baseline escape threshold (ET) prior to IoN-CCI surgery (Pre-IC), baseline ET after IoN-CCI surgery (BL Post-IC); and ET at weeks 2-20 post-varenicline dose are shown in FIG. 3A. IC+Var group 6 animals received IoN-CCI surgery followed by oral varenicline treatment. AAV only group 7 animals received AAV5-m-α7-GlyR LGIC (6×10⁸ vg) followed by IoN-CCI surgery (but no varenicline treatment). AAV/IC+Var group 10 animals received AAV5-m-α7-GlyR LGIC (2×10⁹ vg) followed by IoN-CCI surgery and varenicline treatment 1 month post AAV5-m-α7-GlyR LGIC injection and IC/AAV+Var group 4 animals received IoN-CCI surgery followed by AAV5-m-α7-GlyR LGIC (2×10⁹ vg) and varenicline treatment 1 month post AAV5-m-α7-GlyR LGIC injection.

Male and female animals treated with the combination of AAV5-m-α7-GlyR LGIC and varenicline demonstrated significantly improved ET regardless of whether the AAV5-m-α7-GlyR LGIC injection occurred before or after IoN-CCI surgery. No sex differences in ET were detected throughout the study (FIG. 3A). Further, when the results for each group were averaged between males and females, a significant improvement in mechanical allodynia was observed in all animals receiving AAV5-m-α7-GlyR LGIC and varenicline compared control groups 5, 6 and 7. The improvement in mechanical allodynia in AAV5-m-α7-GlyR LGIC and varenicline treated animals reached approximately 50-60% of naïve control levels (FIG. 3B).

### Example 4

### Determination of Effective Dose

Since no differences were observed between male and female animals for subsequent analysis, male and female data were averaged. Three different doses of AAV5-m-α7-GlyR LGIC (2×10⁸, 6×10⁸, and 2×10⁹ vg/animal) were tested both in animals receiving IoN-CCI surgery before AAV5-m-α7-GlyR LGIC injection (groups 2-4) and animals receiving AAV5-m-α7-GlyR LGIC before IoN-CCI surgery (groups 8-10).

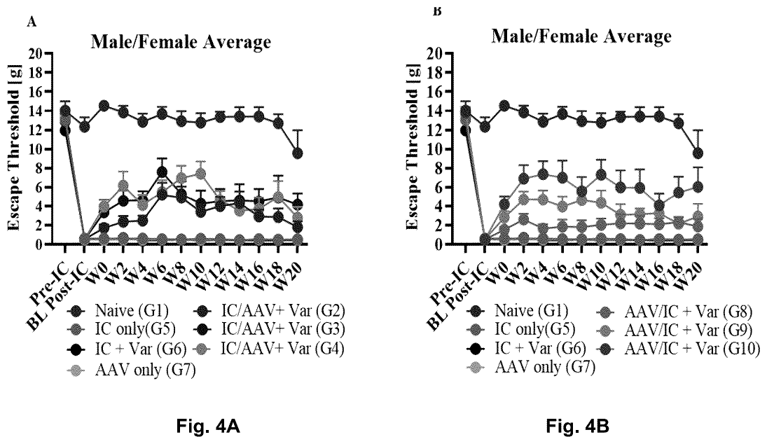

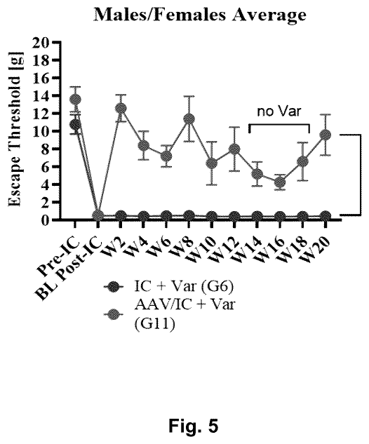

Baseline escape threshold (ET) prior to IoN-CCI surgery (Pre-IC), baseline ET after ION-CCI surgery (BL Post-IC); and ET at weeks 0-20 post-varenicline dose are shown in FIGS. 4A and 4B. Naïve (G1) animals did not receive IoN-CCI surgery, AAV5-m-α7-GlyR LGIC, or varenicline dosing. IC only group 5 animals received IoN-CCI only (without AAV5-m-α7-GlyR LGIC or varenicline). IC+Var group 6 animals received IoN-CCI surgery and varenicline (but no AAV5-m-α7-GlyR LGIC injections). AAV only group 7 animals received AAV5-m-α7-GlyR LGIC (6×10⁸ vg) followed by IoN-CCI surgery (but no varenicline treatment).

IC/AAV+Var group 2 animals received IoN-CCI surgery followed by AAV5-m-α7-GlyR LGIC (2×10⁸ vg) injection (FIG. 4A). IC/AAV+Var group 3 animals received ION-CCI surgery followed by AAV5-m-α7-GlyR LGIC (6×10⁸ vg) and varenicline 1 month post AAV5-m-α7-GlyR LGIC injection (FIG. 4A). IC/AAV+Var group 4 received IoN-CCI surgery followed by AAV5-m-α7-GlyR LGIC (2×10⁹ vg) injection and varenicline 1 month post AAV5-m-α7-GlyR LGIC injection (FIG. 4A).

AAV/IC+Var group 8 animals received AAV5-m-α7-GlyR LGIC (2×10⁸ vg) followed by IoN-CCI surgery and varenicline 1 month post AAV5-m-α7-GlyR LGIC injection (FIG. 4B). AAV/IC+Var group 9 animals received AAV5-m-α7-GlyR LGIC (6×10⁸ vg) followed by IoN-CCI surgery and varenicline 1 month post AAV5-m-α7-GlyR LGIC injection (FIG. 4B). AAV/IC+Var group 10 animals received AAV5-m-α7-GlyR LGIC (2×10⁹ vg) followed by IoN-CCI surgery and varenicline 1 month post AAV5-m-α7-GlyR LGIC injection (FIG. 4B).

AAV5-m-α7-GlyR LGIC in combination with varenicline significantly improved the ET at all doses tested in both animals receiving AAV5-m-α7-GlyR LGIC before or after the ION-CCI surgery (FIGS. 4A and 4B). There were no significant differences between AAV5-m-α7-GlyR LGIC dose groups in both paradigms.

All groups (except Group 12) receiving AAV5-m-α7-GlyR LGIC injections into the trigeminal ganglion in this study were allowed to express the m-α7-GlyR LGIC transgene for 1 month prior to varenicline dosing and efficacy evaluation. However, animals in Group 12 were injected with AAV5-m-α7-GlyR LGIC at 2×10⁹ vg/animal and were allowed 5 months for m-α7-GlyR LGIC transgene to be expressed before IoN-CCI surgery. In this group, varenicline was dosed for 2 weeks beginning 2 weeks following Ion-CCI surgery. To examine the impact of the duration of transgene expression on ET, groups 4, 10, and 12 group (all receiving 2×10⁹ vg/animal; ION-CCI/AAV5-m-α7-GlyR LGIC and AAV5-m-α7-GlyR LGIC/IoN-CCI paradigms) were compared. While after 2 weeks of varenicline dosing, animals in group 12 (m-α7-GlyR LGIC transgene expressed for 5 months prior to varenicline dosing) performed slightly better than animals in groups 4 and 10 (8.6 g ET in Group 12 vs. 6.9 and 6.1 g ET for Groups 4 and 10, respectively), there were no significant differences between all three groups (data not shown). All 3 AAV5-m-α7-GlyR LGIC+varenicline groups performed significantly better than all control groups. In addition, neither varenicline alone nor AAV5-m-α7-GlyR LGIC alone (groups 6 and 7 respectively) improved pain sensitivity (data not shown).

### Example 5

### Long Term Efficacy

The potential for a long-lasting effect of chronic varenicline dosing was evaluated in IoN-CCI rats. Animals in group 11 were injected with AAV5-m-α7-GlyR LGIC 2 weeks prior to IoN-CCI surgery and daily varenicline dosing (0.1 mg/kg) was initiated 1 month post m-α7-GlyR LGIC transgene expression. Rats were treated with varenicline for 3 months, after which the treatment was removed for 1 month, then restarted for the remainder of the study (1 month) (FIG. 5).

IC+Var group 6 animals received IoN-CCI surgery and varenicline (but no AAV5-m-α7-GlyR LGIC) and AAV/IC+Var group 11 animals received AAV5-m-α7-GlyR LGIC (2×10⁹ vg) followed by IoN-CCI surgery and intermittent varenicline (3 months on, 1 month off, 1 month on) treatment (FIG. 5).

Varenicline in combination with AAV5-m-α7-GlyR LGIC significantly increased the escape threshold (ET) throughout the 20 weeks of efficacy assessments (FIG. 5). ET decreased during weeks 14-18 when varenicline was removed (5.2, 4.2 and 6.6 g ET during weeks 14, 16 and 18, respectively [male data only]), and increased again with varenicline re-initiation (9.6 g at week 20 [male data only]) (FIG. 5).

In rodents, the m-α7-GlyR LGIC had no constitutive activity and required the presence of exogeneous activator molecule varenicline for functional activity. In addition, the activation by varenicline of m-α7-GlyR LGICs expressed in mouse cortical neurons resulted in a reversible inhibition of evoked neuronal activity (data not shown). Sustained exposure of the m-α7-GlyR ion channels to varenicline (15 nM) for 18 days in cultured hippocampal neurons did not result in tachyphylaxis or down regulation of the expressed m-α7-GlyR LGICs (data not shown).

### Example 6

### Expression Levels of Modified LGIC in Trigeminal Ganglia

Animals were euthanized at the end of their respective time points to determine AAV5-m-α7-GlyR LGIC vector copy numbers and m-α7-GlyR LGIC mRNA expression (by reverse-transcription quantitative polymerase chain reaction [RT-qPCR]) in the trigeminal ganglia.

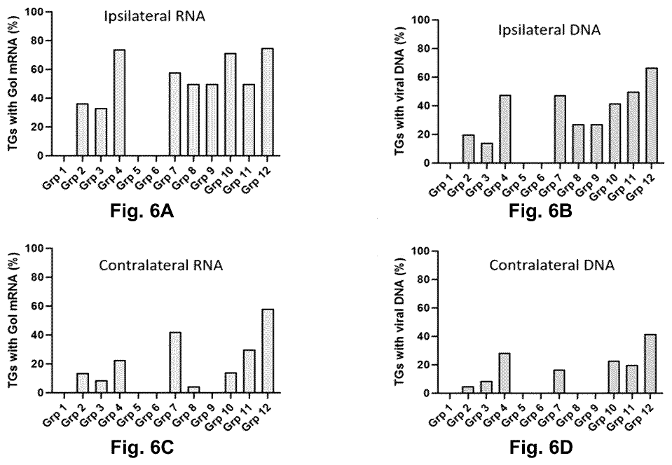

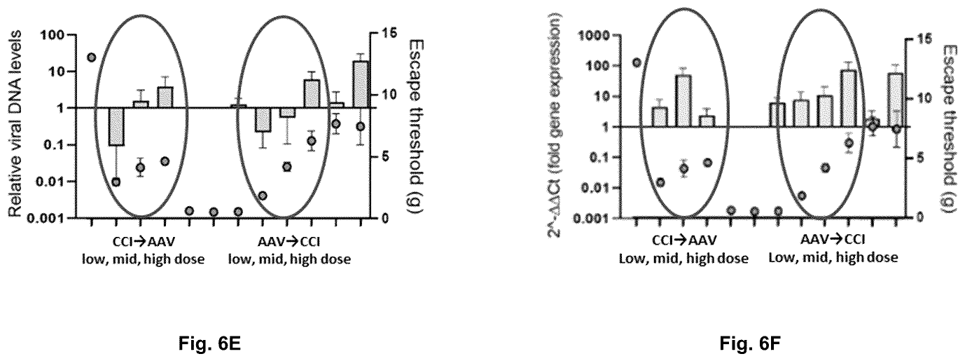

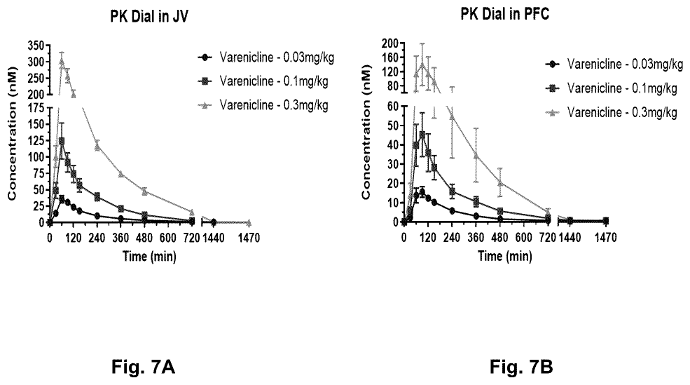

To compare average gene expression of all groups for all time points, the 2-, 4-and 6-month data groups were combined. A dose dependent effect was evident with groups 8-10 (low to high) for both mRNA and DNA and with groups 2-4 (low to high) in the DNA data (FIGS. 6A and 6B). Results showed lower m-α7-GlyR LGIC gene expression and viral DNA on the contralateral side (FIGS. 6C and 6D). Comparison of AAV5-m-α7-GlyR LGIC injected groups 2, 3, 4, 7, 8, 9, 10, and 11 with a one-way ANOVA with Tukey's multiple comparisons showed no significant differences in mRNA or DNA levels on the ipsilateral side.

To investigate the effect of dose on detection of mRNA and AAV5-m-α7-GlyR LGIC viral DNA, the proportion of positive samples were compared. A dose effect was detected on the ipsilateral side for both DNA and m-α7-GlyR LGIC mRNA (FIGS. 6A and 6B).

In summary, AAV5-m-α7-GlyR LGIC+varenicline demonstrated a dose-dependent reversal in mechanical allodynia regardless of treatment paradigm (IoN-CCI before or after AAV5-m-α7-GlyR LGIC injections). The ET significantly improved in both male and female IoN-CCI rats with no significant differences in ET between sexes. While longer expression of m-α7-GlyR LGIC mRNA showed a slightly higher ET, there was no significant differences in efficacy compared to shorter transgene expression duration.

Chronic varenicline (daily dosing for 3 months) treatment in animals injected with AAV5-m-α7-GlyR LGIC demonstrated a durable impact on ET even when varenicline dosing was withdrawn from animals (FIG. 5). There was a decrease in efficacy during the varenicline washout period, but it did not reach baseline levels and efficacy returned once treatment was initiated 4 weeks later (FIG. 5).

Analysis of relative levels of AAV5-m-α7-GlyR LGIC viral DNA and m-α7-GlyR LGIC mRNA demonstrated a dose-dependent DNA/mRNA expression in the injected trigeminal ganglia (FIGS. 6A and 6B). While there were detectable levels of either viral DNA or the m-α7-GlyR LGIC mRNA in uninjected (contralateral) trigeminal ganglia, the levels were significantly lower that on the injected side (FIGS. 6C and 6D compared to 6A and 6B).

Comparison of escape threshold with AAV5-m-α7-GlyR LGIC viral DNA levels (FIG. 6E) and m-α7-GlyR LGIC RNA levels (FIG. 6F) demonstrated increasing escape thresholds with increased levels of AAV5-m-α7-GlyR LGIC viral DNA and m-α7-GlyR LGIC RNA expression. Varenicline levels in all animals receiving treatment were within predictable levels at the time of sample collection.

### Example 7

### Safety Pharmacology

Evaluation of CNS function (measured using a functional observational battery [FOB] test) of animals treated with either AAV5-m-α7-GlyR LGIC (with or without varenicline), or diluent (not receiving AAV5-m-α7-GlyR LGIC but treated with either the diluent/vehicle or varenicline) were included in 6-month rat toxicology and biodistribution studies.

AAV5-m-α7-GlyR LGIC alone (2×10⁸ to 1.96×10¹⁰ vg/animal, delivered in 2 μL) or in combination with 1 mg/kg varenicline administered by oral gavage daily starting 1 month post AAV5-m-α7-GlyR LGIC administration did not differ in FOB observations from the vehicle-treated control rats and rats dosed with varenicline only (data not shown). Overall, there were no AAV5-m-α7-GlyR LGIC, varenicline or AAV5-m-α7-GlyR LGIC+varenicline related changes in the neurological assessments at the 5-or 6-month time points.

### Example 8

### Pharmakokinetics and Drug Metabolism

Since blood PK profile for varenicline in humans at doses higher than those used in the AAV5-m-α7-GlyR LGIC+varenicline combination treatment had previously been established, levels of varenicline were measured in blood as well as brain and CSF following single and/or repeat oral administration without and/or subsequent to AAV5-m-α7-GlyR LGIC administration. Additionally, the tissue biodistribution of AAV5-m-α7-GlyR LGIC after a single trigeminal ganglion injection via the percutaneous route was evaluated over the course of 6 months.

Sixteen male rats were administered varenicline orally via gavage at doses of 0.03, 0.1 and 0.3 mg/kg. Concentrations of varenicline were evaluated in the blood, cerebral spinal fluid (CSF) and prefrontal cortex (PFC) at different time points over the course of 24 hours.

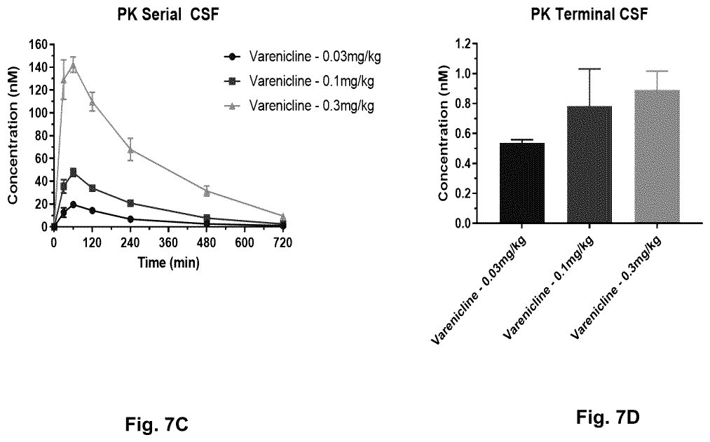

Concentrations of varenicline peaked at 60 minutes in blood, at 90 minutes in PFC, and at 60 minutes in CSF following a single oral administration (FIGS. 7A, 7B, and 7C). Free varenicline drug levels were maintained at or above the EC₅₀ value for varenicline at m-7α-GlyR LGIC (1.6±0.1 nM) for the 0.1 and 0.3 mg/kg doses for up to 12-hours post dosing (FIGS. 7A, 7B, and 7C).

### Example 9

### 28-day Ascending Dose-Ranging Toxicity and Biodistribution in Rats

A 28-day biodistribution/toxicity study was conducted. Briefly, AAV5-m-α7-GlyR LGIC (6×10⁷, 2×10⁸, 6×10⁸, 2×10⁹, 6×10⁹, 2×10¹⁰ vg) or buffer was injected into the trigeminal ganglion on Day 1, followed by a daily oral gavage (starting 1 day after AAV5-m-α7-GlyR LGIC administration) of either sterile water for injection, USP (groups 1 and 8), or varenicline 1 mg/kg p.o. (groups 2 through 7 and 9). Blood samples for varenicline PK analysis were collected prior to dosing, at 2 and 6 hours after dosing on day 2 (the first day of varenicline dosing) and at necropsy (24 hours after the last varenicline dose, day 7 or day 28±1 days).

Of the time points analyzed, varenicline levels were highest at 2 hours post-dose (average of ˜480 nM across groups) and decreased by approximately 50% at 6 hours post-dose and further to an average of 4.4 nM by 24 hours post final dose, which is above the EC₅₀ value of varenicline at the m-α7-GlyR LGIC (FIG. 7A). There were no sex differences in plasma varenicline levels, and AAV5-m-α7-GlyR LGIC had no impact on varenicline levels in any treatment group.

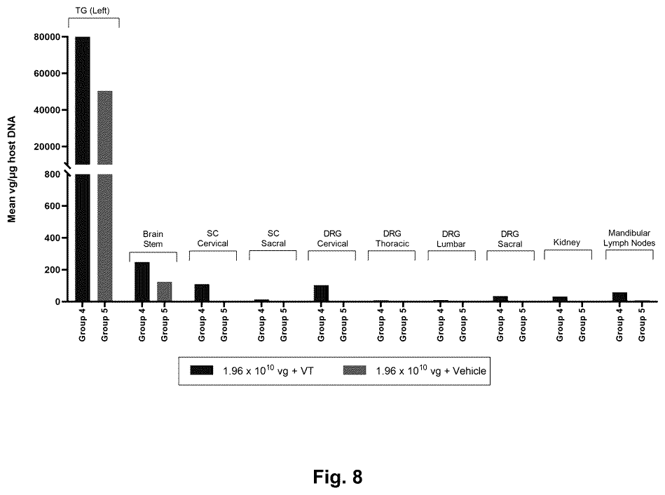

Comparison of the mean tissue-specific AAV5-m-α7-GlyR LGIC vector levels revealed a dose-dependent increase in genomic DNA levels. AAV5-m-α7-GlyR LGIC vector levels were significantly higher in the trigeminal ganglion compared to spinal cord and DRG tissues 7 days and 28 days post AAV5-m-α7-GlyR LGIC injection (FIG. 8). The comparison of the mean tissue-specific AAV5-m-α7-GlyR LGIC vector levels revealed a dose-dependent increase in genomic DNA levels (see FIG. 6B).

The highest vector copy numbers were observed in the injected trigeminal ganglion (450 to 682 copies in the low-dose cohort; 1.02×10⁵ to 1.67×10⁶ copies in the high-dose cohort). In blood, vector genome copies were either absent or very low (undetected in the low-dose cohort; 835 copies on Day 8 to 2 copies on Day 29 in the high-dose cohort). Vector genome copies were absent or at very low levels in the spinal cord (undetected in the low-dose cohort; 163 to 5406 copies in the high-dose cohort) and DRG (undetected in the low-dose cohort; 118 to 564 copies in the high-dose cohort) (FIG. 8). At the highest dose, vector biodistribution in the spinal cord and DRG was 0.3% and 0.03%, respectively, of the amount in the trigeminal ganglion (FIG. 8).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. A nucleic acid having at least 90%, at least 95%, at least 98%, at least 99%, or 100% identity to SEQ ID NO: 4, SEQ ID NO: 5, SEQ ID NO: 6, or SEQ ID NO: 7.

2.-6. (canceled)

7. A polynucleotide comprising:
(i) a 5′ ITR;
(ii) a human synapsin promoter;
(iii) a transgene sequence encoding a Ligand Gated Ion Channel (LGIC) comprising a human a7-nicotinic acetylcholine receptor Ligand Binding Domain (a7-nAChR LBD) and a Glycine Receptor Ion Pore Domain (GlyR IPD);
(iv) a polyA sequence; and
(v) a 3′ ITR.

8. (canceled)

9. The polynucleotide of claim 7, further comprising an intron sequence located between the human synapsin promoter and the transgene sequence encoding the LGIC, wherein:
(i) the 5′ ITR comprises a sequence having at least 90%, 95%, 98%, 99%, or 100% identity to SEQ ID NO: 10;
(ii) the human synapsin promoter comprises a sequence having at least 90%, 95%, 98%, 99% or 100% identity to SEQ ID NO: 11;
(iii) the transgene sequence encodes a polypeptide of SEQ ID NO: 1;
(iv) the poly A sequence comprises a sequence having at least 90%, 95%, 98%, 99%, or 100% identity to SEQ ID NO: 3;
(v) the 3′ ITR comprises a sequence having at least 90%, 95%, 98%, 99%, or 100% identity to SEQ ID NO: 13; and
(vi) the intron comprises a sequence having at least 90%, 95%, 98%, 99%, or 100% identity to SEQ ID NO: 12.

10.-11. (canceled)

12. An expression vector comprising the nucleic acid of claim 1.

13. The expression vector of claim 12, wherein the expression vector is a viral expression vector.

14. The expression vector of claim 13, wherein the vector is an adeno-associated virus (AAV) expression vector.

15. A cell comprising the expression vector of claim 12, wherein the cell is an isolated cell in culture.

16. (canceled)

17. An AAV particle comprising the expression vector of claim 12 and an AAV capsid protein.

18.-23. (canceled)

24. The AAV particle of claim 17, wherein the AAV capsid protein comprises an amino acid sequence that has at least 90%, at least 95%, at least 97%, at least 98%, at least 99% or 100% sequence identity to SEQ ID NO: 9.

25. A composition comprising a nucleic acid of claim 1.

26.-40. (canceled)

41. A method of treating a disease or condition caused by hyperexcitability of the trigeminal nerve, the method comprising administering the composition of claim 25 to a subject in need thereof.

42. The method of claim 41, wherein the disease or condition caused by hyperexcitability of the trigeminal nerve is trigeminal neuralgia, a trigeminal autonomic cephalgia, an episodic cluster headache, a chronic cluster headache; trigeminal deafferation pain; burning mouth syndrome; or post-traumatic trigeminal neuropathic pain.

43. The method of claim 41, wherein the disease or condition is SUNCT or SUNA.

44. The method of claim 42, wherein the disease or condition is trigeminal neuralgia.

45. The method of claim 41, further comprising administering a small molecule agonist to the subject.

46. The method of claim 45, wherein the small molecule agonist is varenicline.

47.-48. (canceled)

49. The method of claim 41, wherein the composition is administered by percutaneous injection.

50.-51. (canceled)

52. The method of claim 49, wherein the method comprises administering about 2×10⁹ to about 2×10¹¹ AAV particles by percutaneous injection into a trigeminal ganglion.

53.-56 (canceled)

57. The method of claim 41, wherein the subject has acute trigeminal neuralgia.

58. The method of claim 41, wherein the subject has chronic trigeminal neuralgia.

59.-65 (canceled)


SEQUENCE LISTING (13 sequences)

SEQ ID NO: 1 - protein, 431 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVGVNSSGHCLYLPPGIFKSSCYIDVRWFPFDVQHCKLKFGSWSYGGWSLDLQMQEADISGYIPNGEWDLVGIPGKRSERFYECCKEPFPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 2 - DNA, 1296 bp, synthetic construct
  source 1..1296: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttatgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaa

SEQ ID NO: 3 - DNA, 517 bp, synthetic construct
  source 1..517: mol_type = other DNA
gtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccc

SEQ ID NO: 4 - DNA, 1821 bp, synthetic construct
  source 1..1821: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttatgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaagcggccgcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccc

SEQ ID NO: 5 - DNA, 1983 bp, synthetic construct
  source 1..1983: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacaggtgatgatatcactagtgctagcgccaccatgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttatgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaagcggccgcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccc

SEQ ID NO: 6 - DNA, 2473 bp, synthetic construct
  source 1..2473: mol_type = other DNA
tgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccgttggatcaggtaagtatcaaggttacaagacaggtttaaggagaccaatagaaactgggcttgtcgagacagagaagactcttgcgtttctgataggcacctattggtcttactgacatccactttgcctttctctccacaggtgatgatatcactagtgctagcgccaccatgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttatgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaagcggccgcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccc

SEQ ID NO: 7 - DNA, 2999 bp, synthetic construct
  source 1..2999: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatccgagctccagtgtgctggaattcgcccttttcagtatttaaattgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccgttggatcaggtaagtatcaaggttacaagacaggtttaaggagaccaatagaaactgggcttgtcgagacagagaagactcttgcgtttctgataggcacctattggtcttactgacatccactttgcctttctctccacaggtgatgatatcactagtgctagcgccaccatgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttatgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaagcggccgcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcccgttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 5446 bp, synthetic construct
  source 1..5446: mol_type = other DNA
agcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatccgagctccagtgtgctggaattcgcccttttcagtatttaaattgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccgttggatcaggtaagtatcaaggttacaagacaggtttaaggagaccaatagaaactgggcttgtcgagacagagaagactcttgcgtttctgataggcacctattggtcttactgacatccactttgcctttctctccacaggtgatgatatcactagtgctagcgccaccatgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttatgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaagcggccgcgtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcccgttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 9 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 10 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 11 - DNA, 480 bp, synthetic construct
  source 1..480: mol_type = other DNA
tgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagcc

SEQ ID NO: 12 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 13 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag